(19) United States
(12) Patent Application Publication
(10) Pub. No.: US 2013/0216579 A1
(43) Pub. Date: Aug. 22, 2013

(54) METHODS AND COMPOSTITIONS FOR GENE EDITING OF A PATHOGEN

(71) Applicants: Sangamo BioSciences, Inc., (US); The Trustees of Columbia University in the City of New York, (US)

(72) Inventors: David Fidock, New York, NY (US); Fyodor Urnov, Richmond, CA (US)

(73) Assignees: The Trustees of Columbia University in the City of New York, New York, NY (US); Sangamo BioSciences, Inc., Richmond, CA (US)

(21) Appl. No.: 13/748,303

(22) Filed: Jan. 23, 2013

Related U.S. Application Data
(60) Provisional application No. 61/589,734, filed on Jan. 23, 2012.
(60) Provisional application No. 61/692,182, filed on Aug. 22, 2012.

(51) Int. Cl. C12N 15/87; C12N 9/22; C07K 14/47

(52) U.S. Cl. CPC C12N 15/87; C07K 14/4702; C12N 9/22
USPC 424/272.1; 530/324; 536/23.5; 435/199; 435/320.1; 435/258.2; 435/471

(57) ABSTRACT

Disclosed herein are methods and compositions for genome editing of the malarial parasite Plasmodium, and for the use of the edited Plasmodium in the development of vaccines and therapeutics.

### CROSS-REFERENCED TO RELATED APPLICATIONS

The present application claims the benefit of U.S. Provisional Application Nos. 61/589,734 filed Jan. 23, 2012 and U.S. Provisional Application 61/692,182 filed Aug. 22, 2012, the disclosures of which are hereby incorporated by reference in its entirety.

### TECHNICAL FIELD

The present disclosure is in the fields of genome editing and vaccine production.

### BACKGROUND

Malaria has affected human development for thousands of years. Although it has apparently been eradicated in some parts of the world, approximately 40 percent of the human population lives in malarial regions. In 2010, the World Health Organization reported three hundred million new cases, and more than 750,000 deaths in that year alone (see Winzeler (2008) Nature 455 p. 751, and Butler et al (2011) Cell Host and Microbe 9 p. 451). Recent reductions in the global burden of disease, brought about by coordinated malaria control efforts reliant on access to first-line artemisinin-based combination therapies and anti-mosquito measures, are at threat of succumbing once again to resistance. This is evidenced by signs of weakening efficacy of artemisinins in southeast Asia. The disease is caused generally by four species of Plasmodium including Plasmodium falciparum, P. vivax, P. ovale and P. malariae and is transmitted through a bite from an infected female Anopheles mosquito. Plasmodium is a protozoan that shares evolutionary ties with other parasites that infect humans and/or livestock such as Babesia, Haemoproteus, and Leucocytozoon.

Part of the difficulty for developing malarial treatments arises from the parasite's complex life cycle. In brief, malaria is transmitted by the mosquito's bite, which deposits Plasmodium sporozoites into the blood stream. A single bite may deposit as few as ten or up to hundreds of the sporozoites into the host. The sporozoites make their way to the liver and form parasitophorous vacuoles in the individual hepatocytes. When in these vacuoles, the parasites may remain dormant as hypnozoites or develop into merozoites. The merozoite-filled vacuoles detach from the liver cells and enter the liver sinusoid where the merozoites are released and infect erythrocytes. Some of the parasites then differentiate into male and female gametocytes that are then taken up by another mosquito during a subsequent bite. Inside the mosquito, the gametocytes become activated gametes that fuse and become a short-lived diploid form called an ookinete. These ookinetes migrate into the mid-gut wall of the mosquito and form an oocyst. Following meiosis in the oocyst, sporozoites are formed that, following rupture of the oocyst, migrate to the mosquito's salivary gland, ready to initiate another cycle.

For a human host, symptoms appear during the erythrocyte infection stage and these can potentially be fatal. The well-known cyclical fevers may correlate to rupture of, and then reinfection of, fresh host red blood cells by the newly released parasites. The liver stage however appears to be asymptomatic. Ideally, a therapeutic against malaria would be effective against both the liver and blood stages of the disease in order to remove all reservoirs from the host. Most malaria treatments used today target the blood stage, and resistance to these drugs is starting to emerge (see Derbyshire et al, (2011) PLoS Pathogens 2011 September; 7(9):e1002178). High-throughput screens have identified small molecules capable of inhibiting pathogen enzyme targets such as histone deacetylase, dihydroorotate dehydrogenase and dihydrofolatereductase, but have not been useful for human therapeutics due to a lack of species specificity by these compounds (Derbyshire, ibid). In fact, most therapeutics currently in use for malaria are derived from compounds that have been known for hundreds of years.

Anti-malarial vaccines have generally focused on the blood cell form of the parasite, but thus far have not been highly effective. It may be that the liver stage of the disease would be a more successful target than the blood stage. The number of parasites that infect the liver is several orders of magnitude less that the number found in the blood during the blood stage, and so inhibiting the disease in the initial phases may be a successful route to inhibition of the lifecycle.

Genomics holds enormous potential for a new era of human therapeutics. These methodologies will allow treatment for conditions that heretofore have not been addressable by standard medical practice. Gene therapy can include the many variations of genome editing techniques such as disruption or correction of a gene locus, and insertion of an expressible transgene that can be controlled either by a specific exogenous promoter fused to the transgene, or by the endogenous promoter found at the site of insertion into the genome. Genetic engineering also holds promise in the development of models for identification of more useful anti-malarials, and for development of new and highly specific vaccines. However, despite sequencing the entire Plasmodium genome, the use of these revolutionary technologies has thus far not yielded successful malarial therapeutics or vaccines. Approximately 50% of the Plasmodium genome encodes open reading frames with unknown identity or function, thus it is difficult to develop compounds to specifically inhibit their gene products. In addition, the machinery for non-homologous end-joining, which is often leveraged in metazoan organisms to produce nuclease-mediated gene disruptions, is notably absent in the P. falciparum genome (that for example lacks Ku70/80 and DNA ligase IV). Homology-directed recombination, which constitutes the alternative pathway of DSB repair, has also been found to be exceptionally inefficient in this parasite.

Thus, there is an urgent need to develop new anti-malarial therapeutics and to develop novel vaccines to arrest the spread of the disease worldwide.

### SUMMARY

Disclosed herein are methods and compositions for genome editing of Plasmodium, including, but not limited to: cleaving of a Plasmodium gene which in turn results in targeted alteration (insertion, deletion and/or substitution mutations) of the Plasmodium gene; targeted introduction into a Plasmodium gene of non-endogenous nucleic acid sequences; the partial or complete inactivation of Plasmodium genes; and/or methods of inducing homology-directed repair at a Plasmodium gene locus. Thus, the methods and compositions described herein can be used to generate anti-malarial therapeutics (e.g., vaccines) as well as for creating models to identify novel and effective anti-malaria therapeutics.

In one aspect, described herein is a method of modifying, using an engineered nuclease, a Plasmodium gene (e.g., an endogenous Plasmodium gene) in a Plasmodium pathogen. In certain embodiments, the Plasmodium gene is Dxr (PlasmoDB ID: PF14_{—}0641), Elo1 (PFA0455c), pfcrt (MAL7P1.27), pfmdr1 (PFE1150w) and/or LipB (MAL8P1.37). In certain embodiments, two ZFNs that bind to first and second target sites in a Plasmodium gene and form a dimer upon binding are used to cleave the Plasmodium gene between the first and second target sites. Furthermore, any of the methods described herein may further comprise introducing into the cell an exogenous sequence wherein cleavage by the ZFN(s) results in integration (insertion) of an exogenous sequence into the Plasmodium gene. In another aspect, described herein is a zinc-finger protein (ZFP) that binds to target site in a Plasmodium gene in a genome, wherein the ZFP comprises one or more engineered zinc-finger binding domains. In certain embodiments, the ZFP comprises 5 or 6 zinc fingers ordered F1 to F5 or F1 to F6, which zinc fingers comprise the recognition helix region sequences shown in a single row of Table 1. In one embodiment, the ZFP is fused to a cleavage (nuclease) domain (or cleavage half-domain) to form a zinc-finger nuclease (ZFN) that cleaves a target genomic region of interest, for example as a dimer Cleavage domains and cleavage half domains can be obtained, for example, from various restriction endonucleases and/or homing endonucleases. In one embodiment, the cleavage half-domains are derived from a Type IIS restriction endonuclease (e.g., Fok I). In certain embodiments, the zinc finger domain recognizes a target site in a Dxr, Elo1, pfcrt, pfmdr1 or LipB Plasmodium gene.

The ZFN(s) as described herein may bind to and/or cleave a Plasmodium gene within the coding region of the gene or in a non-coding sequence within or adjacent to the gene, such as, for example, a leader sequence, trailer sequence or intron, or within a non-transcribed region, either upstream or downstream of the coding region.

In another aspect, described herein is a TALE protein (Transcription activator like effector) that binds to target site in a Plasmodium gene in a genome, wherein the TALE comprises one or more engineered TALE binding domains. In one embodiment, the TALE is a nuclease (TALEN) that cleaves a target genomic region of interest, wherein the TALEN comprises one or more engineered TALE DNA binding domains and a nuclease cleavage domain or cleavage half-domain. Cleavage domains and cleavage half domains can be obtained, for example, from various restriction endonucleases and/or homing endonucleases. In one embodiment, the cleavage half-domains are derived from a Type IIS restriction endonuclease (e.g., Fok I). In certain embodiments, the TALE DNA binding domain recognizes a target site in a Dxr, Elo1 or LipB gene.

The TALEN may bind to and/or cleave a Plasmodium gene within the coding region of the gene or in a non-coding sequence within or adjacent to the gene, such as, for example, a leader sequence, trailer sequence or intron, or within a non-transcribed region, either upstream or downstream of the coding region.

In another aspect, described herein is a polynucleotide encoding one or more the proteins described herein (e.g., ZFPs, ZFNs, TALEs and/or TALENs) described herein. In any of the methods described herein, the polynucleotide encoding the zinc finger nuclease(s) or TALEN(s) can comprise DNA, RNA (e.g., mRNA) or combinations thereof. In certain embodiments, the polynucleotide comprises a plasmid. In other embodiments, the polynucleotide encoding the nuclease comprises mRNA.

In some aspects, the mRNA may be chemically modified (See e.g. Kormann et al, (2011) Nature Biotechnology 29(2):154-157). In another aspect, described herein is an expression vector comprising any of the polynucleotides described herein, including polynucleotides encoding one or more ZFNs or TALENs. In certain embodiments, the expression vector comprises a promoter to which the protein-encoding sequence is operably linked.

In another aspect, described herein is a method for cleaving one or more Plasmodium genes in a cell, the method comprising: (a) introducing, into the cell, one or more polynucleotides encoding one or more ZFNs or TALENs that bind to a target site in the one or more genes under conditions such that the ZFN(s) is (are) or TALENs is (are) expressed and the one or more Plasmodium genes are cleaved.

In another embodiment, described herein is a method for modifying one or more Plasmodium gene sequence(s) in the genome of cell, the method comprising (a) providing a Plasmodium cell, and (b) expressing first and second zinc-finger nucleases (ZFNs) or TALENs in the cell, wherein the first ZFN or TALEN binds to (and/or cleaves) at a first site and the second ZFN or TALEN binds to (and/or cleaves) at a second site, wherein the gene sequence is located between the first and second sites, wherein cleavage at the first and/or second sites results in modification of the gene. Optionally, the cleavage results in insertion of an exogenous sequence (transgene) also introduced into the cell. In other embodiments, gene modification results in a deletion between the first and second sites. The size of the deletion in the gene sequence is determined by the distance between the first and second cleavage sites. Accordingly, deletions of any size, in any genomic region of interest, can be obtained. Deletions of 1, 5, 10, 25, 50, 100, 200, 300, 400, 500, 600, 700, 800, 900, 1,000 nucleotide pairs, or any integral value of nucleotide pairs within this range, can be obtained. In addition deletions of a sequence of any integral value of nucleotide pairs greater than 1,000 nucleotide pairs can be obtained using the methods and compositions disclosed herein. Using these methods and compositions, mutant Plasmodium proteins may be developed, which in turn can be used to study the function of the protein within a cell.

In another aspect, described herein are methods of inactivating a Plasmodium gene in a cell by introducing one or more proteins, polynucleotides and/or vectors into the cell as described herein. In any of the methods described herein the ZFNs and/or TALENs may induce targeted mutagenesis, targeted deletions of cellular DNA sequences, and/or facilitate targeted recombination at a predetermined Plasmodium chromosomal locus. Thus, in certain embodiments, the ZFNs and/or TALENs delete or insert one or more nucleotides into the target gene. In some embodiments, the Dxr, Elo1, pfcrt, pfmdr1 or LipB genes are inactivated by ZFN or TALEN cleavage in the presence of a suitable donor. In other embodiments, a genomic sequence in the target gene is replaced, for example using a ZFN or TALEN (or vector encoding said ZFN or TALEN) as described herein and a “donor” sequence that is inserted into the gene following targeted cleavage with the ZFN or TALEN. The donor sequence (exogenous sequence) may be present in the ZFN or TALEN vector, present in a separate vector or, alternatively, may be introduced into the cell using a different nucleic acid delivery mechanism.

In another aspect provided by the methods and compositions of the invention is the use of cells, cell lines and animals (e.g., transgenic animals) in the screening of drug libraries and/or other therapeutic compositions (i.e., antibodies, structural RNAs, etc.) for use in treatment of an animal afflicted with malaria. Such screens can begin at the cellular level with manipulated Plasmodium cells comprising modified genes, and can progress up to the level of treatment of a whole animal, for example a mouse or rat infected with the rodent malaria species Plasmodium berghei, Plasmodium yoelii or Plasmodium vinckeii. Other animal models include primates infected with the species Plasmodium vivax or Plasmodium knowlesi. In some embodiments, parasites are altered by nuclease-mediated genome engineering. In some aspects, the genome engineering modifies genes involved in resistance to anti-malarials. In some cases, the gene modified is pfcrt and/or pfmdr1. The methods and compositions of the invention provide compositions of genome-engineered parasites that can be used for drug library or other therapeutic reagents screening. In certain embodiments, the methods of screening comprise the steps of: providing a mutant of a single celled Plasmodium organism wherein the mutant is altered in pfcrt and/or pfmdr 1 sequence composition such that the organism has different drug susceptibility properties; and contacting the mutant organism with a compound (e.g., a therapeutic compound) library, and identifying compounds capable of inhibiting growth and/or replication of the parasite. In certain embodiments, the compound includes one or more therapeutic molecules, one or more antibodies, one or more interfering RNAs or the like. A library of compounds may also be used.

In some embodiments of the invention, the methods and compositions are used to make a pharmaceutical composition (e.g., vaccine) for the treatment and/or prevention of malaria in mammals. Specifically, the invention provides reagents and methods for inhibiting Plasmodium invasion and/or replication in cells, especially red blood cells, and vaccines for preventing malaria. In some embodiments, the composition comprises at least one nuclease-modified Plasmodium spp. that is administered to the subject for treatment or prevention of malaria. Plasmodium species relating to the reagents and methods of the invention include but are not limited to Plasmodium falciparum, Plasmodium vivax, Plasmodium malariae, Plasmodium knowlesi and Plasmodium ovale. In some aspects, pathogens are treated with the ZFNs or TALENs of the invention such that one or more genes are inactivated (e.g., Dxr, Elo1, and/or LipB genes). In other embodiments, the invention provides a composition comprising Plasmodium pathogens that are unable to transition to the blood borne stage. Thus, the methods and compositions of the invention provide novel strains of Plasmodium that can be used to treat, prevent and/or control malarial infections caused by this pathogen. These mutant pathogens can then be expanded, and used for vaccine in animals in need thereof.

Some aspects of the invention provide methods for generating an immune response (e.g., vaccinating) a patient, comprising the steps of: providing a mutant of a single celled Plasmodium organism wherein said mutant is deficient in Dxr, Elo 1 and/or LipB activity; and contacting a mammal with said mutant foam. In some embodiments, the parasite is Plasmodium falciparum. In some embodiments the parasite used is either alive or killed in the vaccine. An “immune response” is the development in a subject of a humoral and/or a cellular immune response, typically to an antigen present in the composition of interest. Thus, an immune response may include an immune responses mediated by antibody molecules and/or responses mediated by T-lymphocytes (e.g., cytolytic T-cells, helper T-cells, etc.) and/or other white blood cells. An immune response may be protective (e.g., prevent infection of the subject with malaria) and/or therapeutic (e.g. treat a subject with a malaria infection).

In another aspect, the invention provides kits for generating an immune response against Plasmodium spp., treating and/or preventing malaria comprising a pharmaceutical composition as described herein and, optionally, instructions for use.

A kit, comprising the ZFPs or TALENs of the invention, is also provided. The kit may comprise nucleic acids encoding the ZFPs or TALENs, (e.g. RNA molecules or ZFP or TALEN encoding genes contained in a suitable expression vector), donor molecules, aliquots of the ZFN or TALEN proteins, suitable host cell lines, instructions for performing the methods of the invention, and the like.

These and other aspects will be readily apparent to the skilled artisan in light of disclosure as a whole.

### BRIEF DESCRIPTION OF THE DRAWINGS

FIG. 1, panels A through F, show 2A-linked ZFNs drive disruption of egfp in P. falciparum. FIG. 1A shows that coexpression of 2A-linked mRFP and GFP monomers from a single calmodulin (cam) promoter as evidenced by fluorescence microscopy (lower left panel) and immunoblotting (lower right panel) for GFP. The 2A sequence is indicated in the schematic at the top (SEQ ID NO:15). The arrow indicates the ribosome skip site. “C” indicates control untransfected parasites in the GFP immunoblot. FIG. 1B depicts the strategy used to disrupt egfp integrated at the genomic cg6 locus. The donor plasmid encodes 2A-linked left (ZFN L) and right (ZFN R) ZFNs in addition to egfp homologous regions (egfp 5′, egfp 3′) flanking the ZFN target site (thunderbolt). Repair of the ZFN-induced DSB, via homology-directed repair using the donor as template, yielded an in-frame integration of hdhfr into the egfp locus. FIG. 1C is a panel ofmicrographs showing EGFP expression in the parental line NF54^{EGFP} (top panel) and the recombinant line NF54^{ΔegfpA} (lower panel). Nuclei were stained with Hoechst 33342. FIG. 1D shows a gel of PCR analysis of the ZFN-transfected lines NF54^{ΔegfrA-B3} and the parental line NF54^{EGFP} using the primers indicated in FIG. 1B, bottom illustration (see, also, Table 3). FIG. 1E shows results of Southern blot hybridization of genomic DNA digested with ClaI+BamHI (locations indicated in FIG. 1B) and demonstrates integration of hdhfr in the ZFN-transfected lines (lower panel) and the expected 2 kb size increase at the disrupted egfp locus (upper panel). FIG. 1F depicts results of flow cytometry showing EGFP signal in the indicated ZFN-modified parasite populations.

FIG. 2, panels A to E, depict ZFN-mediated replacement of egfp. FIG. 2A is a schematic of the egfp replacement strategy. ZFNs were expressed from the calmodulin promoter on the pZFN^{egfp}-hdhfr plasmid (ZFN plasmid) and cotransfected with the mrfp-vps4 donor sequence (donor plasmid). Homology-directed repair of the ZFN-induced DSB, using the flanking regions on the donor as template, resulted in replacement of egfp with the mrfp-vps4 fusion construct. FIG. 2B shows fluorescence micrographs showing EGFP and mRFP expression in the parental line NF54^{EGFP} and in post-ZFN bulk culture or a clonal line as indicated. Nuclei were stained with Hoechst 33342. FIG. 2C is a graph showing quantification of parasite fluorescence following ZFN mediated insertion of mRFP-Vps4 in the bulk culture in two independent experiments (n=1042 and n=1032) Each bar shows no fluorescence (gray shading at top of each bar); both EGFP and mRFP fluorescence (black shading underneath no fluorescence on each bar); EGFP fluorescence (light gray shading on each bar); and mRFP fluorescence (dark gray shading at the bottom of each bar) FIG. 2D depicts PCR analysis of parental NF54^{EGFP} and ZFN-transfected parasites for a bulk culture and individual parasite clones. Primer positions are shown in FIG. 2A. FIG. 2E shows Southern blot hybridization of genomic DNA from the indicated parasite lines digested with ClaI+BamHI (FIG. 2A), using an egfp probe (left panel) and a mrfp probe (right panel). Linearized transfection plasmids served as positive controls.

FIG. 3, panels A to D, depict ZFN-driven allelic replacement of pfcrt. FIG. 3A is a schematic depicting pfcrt allelic replacement strategy. The pZFN^{crt}-bsd plasmid encodes pfcrt-specific ZFNs, driven by the calmodulin promoter. The pcrt^{Dd2}-hdhfrdonor plasmid contains the 1.2 kb coding sequence of the Dd2 pfcrt allele, followed by 0.7 kb of the pbcrt 3′ UTR, and the hdhfr selectable marker. These cassettes are flanked by two homology regions: 0.4 kb upstream of the DSB and 1 kb of the pfcrt 3′ UTR. ZFN-driven homology-directed repair yielded the pfcrt-modified GC03^{crt-Dd2}locus. FIG. 3B shows PCR analysis of two independent clones. Primer positions are shown in FIG. 3A. FIG. 3C shows Southern blotting of genomic DNA from the indicated parasite lines digested with SalI+BstBI and probed for hdhfr (black bar in FIG. 3A). The band size (6.7 kb) observed with clones G9 and H6 is consistent with pfcrt replacement (no band). The pcrt^{Dd2}-hdhfr plasmid was linearized with SpeI (8.1 kb). FIG. 3D is a plot showing half-maximal inhibitory concentration (IC₅₀) values for the indicated parasite lines (see Example 4). Asterisks indicate significant difference between the two representative pfcrt allelic replacement clones GC03^{crt-Dd2G9} and GC03^{crt-Dd2H6} and the GC03 parental line (*P=0.0286, Mann-Whitney U test, two-tailed, n=4).

FIG. 4, panels A to C, show ZFN-editing of pfcrt with and without chloroquine selection. FIG. 4A is a schematic depicting pfcrt editing strategy. The calmodulin promoter drives expression of the pfcrt-specific ZFN pairs from plasmids with (pZFN^{crt}-76I-hdhfr) or without (pZFN^{crt}-76I) the selectable marker hdhfr. The homologous donor sequence for DSB repair comprises a fragment of pfcrt stretching 0.4 kb upstream and 0.6 kb downstream of the ZFN target site (thunderbolt). One version of the donor (termed ‘mut1’) is identical to the genomic locus but contains the mutant I76 codon (starred) conferring CQ resistance, and a single nucleotide deletion, T₇versus T₈, in the endogenous 5′ UTR. An alternate donor construct (‘mut2’, not shown) is mutated at the ZFN binding site. Homology-dependent repair of a ZFN-induced DSB leads to incorporation of donor-provided SNPs. FIG. 4B is a bar graph showing half-maximal inhibitory concentration (IC₅₀) values for the indicated parasite lines. The asterisk indicates that the 106/1 parental line is significantly different (P<0.0286, Mann-Whitney U test, n=4, two-tailed) from the gene-edited parasites. FIG. 4C shows chromatograms depicting sequence analysis of genomic and mutt recombinant DNA. The 5′ UTR deletion and the mutations at the ZFN binding site and the CQ resistance-conferring I76 codon are indicated.

### DETAILED DESCRIPTION

Disclosed herein are methods and compositions for creating models for identification of novel and effective anti-malaria therapeutics, as well as methods and compositions for preventing malaria. The compositions and methods described herein can be used for genome editing of Plasmodium, including, but not limited to: cleaving of a Plasmodium gene resulting in targeted alteration (insertion, deletion and/or substitution mutations) in the targeted gene, targeted introduction into a Plasmodium gene of non-endogenous nucleic acid sequences, the partial or complete inactivation of a Plasmodium gene; and methods of inducing homology-directed repair at a Plasmodium gene locus.

General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley &Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

### DEFINITIONS

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (K_{d}) of 10⁻⁶ M⁻¹ or lower. “Affinity” refers to the strength of binding: increased binding affinity being correlated with a lower K_{d}.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP.

A “TALE DNA binding domain” or “TALE” is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. See, e.g., U.S. Patent Publication No. 20110301073, incorporated by reference herein in its entirety.

Zinc finger binding domains can be “engineered” to bind to a predetermined nucleotide sequence, for example via engineering (altering one or more amino acids) of the recognition helix region of a naturally occurring zinc finger protein. Similarly, TALEs can be “engineered” to bind to a predetermined nucleotide sequence, for example by engineering of the amino acids involved in DNA binding (the RVD region). Therefore, engineered zinc finger proteins or TALE proteins are proteins that are non-naturally occurring. Non-limiting examples of methods for engineering zinc finger proteins and TALEs are design and selection. A designed protein is a protein not occurring in nature whose design/composition results principally from rational criteria. Rational criteria for design include application of substitution rules and computerized algorithms for processing information in a database storing information of existing ZFP or TALE designs and binding data. See, for example, U.S. Pat. Nos. 6,140,081; 6,453,242; and 6,534,261; see also WO 98/53058; WO 98/53059; WO 98/53060; WO 02/016536 and WO 03/016496.

A “selected” zinc finger protein or TALE is a protein not found in nature whose production results primarily from an empirical process such as phage display, interaction trap or hybrid selection. See e.g., U.S. Pat. No. 5,789,538; U.S. Pat. No. 5,925,523; U.S. Pat. No. 6,007,988; U.S. Pat. No. 6,013,453; U.S. Pat. No. 6,200,759; WO 95/19431; WO 96/06166; WO 98/53057; WO 98/54311; WO 00/27878; WO 01/60970 WO 01/88197 and WO 02/099084.

“Recombination” refers to a process of exchange of genetic information between two polynucleotides. For the purposes of this disclosure, “homologous recombination (HR)” refers to the specialized form of such exchange that takes place, for example, during repair of double-strand breaks in cells via homology-directed repair mechanisms. This process requires nucleotide sequence homology, uses a “donor” molecule to template repair of a “target” molecule (i.e., the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that farms between the broken target and the donor, and/or “synthesis-dependent strand annealing,” in which the donor is used to re-synthesize genetic information that will become part of the target, and/or related processes. Such specialized HR often results in an alteration of the sequence of the target molecule such that part or all of the sequence of the donor polynucleotide is incorporated into the target polynucleotide.

In the methods of the disclosure, one or more targeted nucleases as described herein create a double-stranded break in the target sequence (e.g., cellular chromatin) at a predetermined site, and a “donor” polynucleotide, having homology to the nucleotide sequence in the region of the break, can be introduced into the cell. The presence of the double-stranded break has been shown to facilitate integration of the donor sequence. The donor sequence may be physically integrated or, alternatively, the donor polynucleotide is used as a template for repair of the break via homologous recombination, resulting in the introduction of all or part of the nucleotide sequence as in the donor into the cellular chromatin. Thus, a first sequence in cellular chromatin can be altered and, in certain embodiments, can be converted into a sequence present in a donor polynucleotide. Thus, the use of the terms “replace” or “replacement” can be understood to represent replacement of one nucleotide sequence by another, (i.e., replacement of a sequence in the informational sense), and does not necessarily require physical or chemical replacement of one polynucleotide by another.

In any of the methods described herein, additional pairs of zinc-finger or TALEN proteins can be used for additional double-stranded cleavage of additional target sites within the cell.

In certain embodiments of methods for targeted recombination and/or replacement and/or alteration of a sequence in a region of interest in cellular chromatin, a chromosomal sequence is altered by homologous recombination with an exogenous “donor” nucleotide sequence. Such homologous recombination is stimulated by the presence of a double-stranded break in cellular chromatin, if sequences homologous to the region of the break are present.

In any of the methods described herein, the exogenous sequence (the “donor sequence”) can contain sequences that are homologous, but not identical, to genomic sequences in the region of interest, thereby stimulating homologous recombination to insert a non-identical sequence in the region of interest. Thus, in certain embodiments, portions of the donor sequence that are homologous to sequences in the region of interest exhibit between about 80 to 99% (or any integer therebetween) sequence identity to the genomic sequence that is replaced. In other embodiments, the homology between the donor and genomic sequence is higher than 99%, for example if only 1 nucleotide differs as between donor and genomic sequences of over 100 contiguous base pairs. In certain cases, a non-homologous portion of the donor sequence can contain sequences not present in the region of interest, such that new sequences are introduced into the region of interest. In these instances, the non-homologous sequence is generally flanked by sequences of 50-1,000 base pairs (or any integral value therebetween) or any number of base pairs greater than 1,000, that are homologous or identical to sequences in the region of interest. In other embodiments, the donor sequence is inserted into the genome by non-homologous recombination mechanisms.

Any of the methods described herein can be used for partial or complete inactivation of one or more target sequences in a cell by targeted integration of donor sequence that disrupts expression of the gene(s) of interest. Cell lines with partially or completely inactivated genes are also provided.

Furthermore, the methods of targeted integration as described herein can also be used to integrate one or more exogenous sequences. The exogenous nucleic acid sequence can comprise, for example, one or more genes or cDNA molecules, or any type of coding or non-coding sequence, as well as one or more control elements (e.g., promoters). In addition, the exogenous nucleic acid sequence may produce one or more RNA molecules (e.g., small hairpin RNAs (shRNAs), inhibitory RNAs (RNAis), microRNAs (miRNAs), etc.).

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

A “cleavage half-domain” is a polypeptide sequence which, in conjunction with a second polypeptide (either identical or different) forms a complex having cleavage activity (preferably double-strand cleavage activity). The terms “first and second cleavage half-domains;” “+ and − cleavage half-domains” and “right and left cleavage half-domains” are used interchangeably to refer to pairs of cleavage half-domains that dimerize.

An “engineered cleavage half-domain” is a cleavage half-domain that has been modified so as to form obligate heterodimers with another cleavage half-domain (e.g., another engineered cleavage half-domain). See, also, U.S. Patent Publication Nos. 2005/0064474, 2007/0218528; 2008/0131962 and 20110201055, incorporated herein by reference in their entireties.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value therebetween or thereabove), preferably between about 100 and 1,000 nucleotides in length (or any integer therebetween), more preferably between about 200 and 500 nucleotides in length.

“Chromatin” is the nucleoprotein structure comprising the cellular genome. Cellular chromatin comprises nucleic acid, primarily DNA, and protein, including histones and non-histone chromosomal proteins. The majority of eukaryotic cellular chromatin exists in the form of nucleosomes, wherein a nucleosome core comprises approximately 150 base pairs of DNA associated with an octamer comprising two each of histones H2A, H2B, H3 and H4; and linker DNA (of variable length depending on the organism) extends between nucleosome cores. A molecule of histone H1 is generally associated with the linker DNA. For the purposes of the present disclosure, the teen “chromatin” is meant to encompass all types of cellular nucleoprotein, both prokaryotic and eukaryotic. Cellular chromatin includes both chromosomal and episomal chromatin.

A “chromosome,” is a chromatin complex comprising all or a portion of the genome of a cell. The genome of a cell is often characterized by its karyotype, which is the collection of all the chromosomes that comprise the genome of the cell. The genome of a cell can comprise one or more chromosomes.

An “episome” is a replicating nucleic acid, nucleoprotein complex or other structure comprising a nucleic acid that is not part of the chromosomal karyotype of a cell. Examples of episomes include plasmids and certain viral genomes.

A “target site” or “target sequence” is a nucleic acid sequence that defines a portion of a nucleic acid to which a binding molecule will bind, provided sufficient conditions for binding exist.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer. An exogenous molecule can also be the same type of molecule as an endogenous molecule but derived from a different species than the cell is derived from. For example, a human nucleic acid sequence may be introduced into a cell line originally derived from a mouse or hamster.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “fusion” molecule is a molecule in which two or more subunit molecules are linked, preferably covalently. The subunit molecules can be the same chemical type of molecule, or can be different chemical types of molecules. Examples of the first type of fusion molecule include, but are not limited to, fusion proteins (for example, a fusion between a ZFP or TALE DNA-binding domain and one or more activation domains) and fusion nucleic acids (for example, a nucleic acid encoding the fusion protein described supra). Examples of the second type of fusion molecule include, but are not limited to, a fusion between a triplex-forming nucleic acid and a polypeptide, and a fusion between a minor groove binder and a nucleic acid.

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are presented elsewhere in this disclosure.

A “gene,” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” of gene expression refers to a change in the activity of a gene. Modulation of expression can include, but is not limited to, gene activation and gene repression. Genome editing (e.g., cleavage, alteration, inactivation, random mutation) can be used to modulate expression. Gene inactivation refers to any reduction in gene expression as compared to a cell that does not include a ZFP or TALEN as described herein. Thus, gene inactivation may be partial or complete.

A “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

“Eukaryotic” cells include, but are not limited to, fungal cells (such as yeast), plant cells, animal cells, mammalian cells and human cells (e.g., T-cells).

“Secretory tissues” are those tissues in an animal that secrete products out of the individual cell into a lumen of some type which are typically derived from epithelium. Examples of secretory tissues that are localized to the gastrointestinal tract include the cells that line the gut, the pancreas, and the gallbladder. Other secretory tissues include the liver, tissues associated with the eye and mucous membranes such as salivary glands, mammary glands, the prostate gland, the pituitary gland and other members of the endocrine system. Additionally, secretory tissues may be thought of as individual cells of a tissue type which are capable of secretion.

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

With respect to fusion polypeptides, the term “operatively linked” can refer to the fact that each of the components performs the same function in linkage to the other component as it would if it were not so linked. For example, with respect to a fusion polypeptide in which a ZFP or TALE DNA-binding domain is fused to an activation domain, the ZFP or TALE DNA-binding domain and the activation domain are in operative linkage if, in the fusion polypeptide, the ZFP or TALE DNA-binding domain portion is able to bind its target site and/or its binding site, while the activation domain is able to up-regulate gene expression. When a fusion polypeptide in which a ZFP or TALE DNA-binding domain is fused to a cleavage domain, the ZFP or TALE DNA-binding domain and the cleavage domain are in operative linkage if, in the fusion polypeptide, the ZFP or TALE DNA-binding domain portion is able to bind its target site and/or its binding site, while the cleavage domain is able to cleave DNA in the vicinity of the target site.

A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA-binding function of a polypeptide can be determined, for example, by filter-binding, electrophoretic mobility-shift, or immunoprecipitation assays. DNA cleavage can be assayed by gel electrophoresis. See, Ausubel et al., supra. The ability of a protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and PCT WO 98/44350.

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors.

A “reporter gene” or “reporter sequence” refers to any sequence that produces a protein product that is easily measured, preferably although not necessarily in a routine assay. Suitable reporter genes include, but are not limited to, sequences encoding proteins that mediate antibiotic resistance (e.g., ampicillin resistance, neomycin resistance, G418 resistance, puromycin resistance), sequences encoding colored or fluorescent or luminescent proteins (e.g., green fluorescent protein, enhanced green fluorescent protein, red fluorescent protein, luciferase), and proteins which mediate enhanced cell growth and/or gene amplification (e.g., dihydrofolatereductase). Epitope tags include, for example, one or more copies of FLAG, His, myc, Tap, HA or any detectable amino acid sequence. “Expression tags” include sequences that encode reporters that may be operably linked to a desired gene sequence in order to monitor expression of the gene of interest.

Nucleases

Described herein are compositions, particularly nucleases, which are useful targeting a gene for the insertion of a transgene, for example, nucleases that are specific for albumin. In certain embodiments, the nuclease is naturally occurring. In other embodiments, the nuclease is non-naturally occurring, i.e., engineered in the DNA-binding domain and/or cleavage domain. For example, the DNA-binding domain of a naturally-occurring nuclease may be altered to bind to a selected target site (e.g., a meganuclease that has been engineered to bind to site different than the cognate binding site). In other embodiments, the nuclease comprises heterologous DNA-binding and cleavage domains (e.g., zinc finger nucleases; TAL-effector nucleases; meganuclease DNA-binding domains with heterologous cleavage domains).

A. DNA-Binding Domains

In certain embodiments, the nuclease is a meganuclease (homing endonuclease). Naturally-occurring meganucleases recognize 15-40 base-pair cleavage sites and are commonly grouped into four families: the LAGLIDADG family, the GIY-YIG family, the His-Cyst box family and the HNH family. Exemplary homing endonucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII. Their recognition sequences are known. See also U.S. Pat. No. 5,420,032; U.S. Pat. No. 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue.

In certain embodiments, the nuclease comprises an engineered (non-naturally occurring) homing endonuclease (meganuclease). The recognition sequences of homing endonucleases and meganucleases such as I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII are known. See also U.S. Pat. No. 5,420,032; U.S. Pat. No. 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue. In addition, the DNA-binding specificity of homing endonucleases and meganucleases can be engineered to bind non-natural target sites. See, for example, Chevalier et al. (2002) Molec. Cell 10:895-905; Epinat et al. (2003) Nucleic Acids Res. 31:2952-2962; Ashworth et al. (2006) Nature 441:656-659; Paques et al. (2007) Current Gene Therapy 7:49-66; U.S. Patent Publication No. 20070117128. The DNA-binding domains of the homing endonucleases and meganucleases may be altered in the context of the nuclease as a whole (i.e., such that the nuclease includes the cognate cleavage domain) or may be fused to a heterologous cleavage domain.

In other embodiments, the DNA-binding domain comprises a naturally occurring or engineered (non-naturally occurring) TAL effector DNA binding domain. See, e.g., U.S. Patent Publication No. 20110301073, incorporated by reference in its entirety herein. The plant pathogenic bacteria of the genus Xanthomonas are known to cause many diseases in important crop plants. Pathogenicity of Xanthomonas depends on a conserved type III secretion (T3S) system which injects more than 25 different effector proteins into the plant cell. Among these injected proteins are transcription activator-like effectors (TALE) which mimic plant transcriptional activators and manipulate the plant transcriptome (see Kay et al (2007) Science 318:648-651). These proteins contain a DNA binding domain and a transcriptional activation domain. One of the most well characterized TALEs is AvrBs3 from Xanthomonas campestgris pv. Vesicatoria (see Bonas et al (1989) Mol Gen Genet 218: 127-136 and WO2010079430). TALEs contain a centralized domain of tandem repeats, each repeat containing approximately 34 amino acids, which are key to the DNA binding specificity of these proteins. In addition, they contain a nuclear localization sequence and an acidic transcriptional activation domain (for a review see Schornack S, et al (2006) J Plant Physiol 163(3): 256-272). In addition, in the phytopathogenic bacteria Ralstonia solanacearum two genes, designated brg11 and hpx17 have been found that are homologous to the AvrBs3 family of Xanthomonas in the R. solanacearum biovar 1 strain GMI1000 and in the biovar 4 strain RS1000 (See Heuer et al (2007) Appland Envir Micro 73(13): 4379-4384). These genes are 98.9% identical in nucleotide sequence to each other but differ by a deletion of 1,575 by in the repeat domain of hpx17. However, both gene products have less than 40% sequence identity with AvrBs3 family proteins of Xanthomonas.

Thus, in some embodiments, the DNA binding domain that binds to a target site a Plasmodium gene is an engineered domain from a TAL effector similar to those derived from the plant pathogens Xanthomonas (see Boch et al, (2009) Science 326: 1509-1512 and Moscou and Bogdanove, (2009) Science 326: 1501) and Ralstonia (see Heuer et al (2007) Applied and Environmental Microbiology 73(13): 4379-4384); U.S. Patent Publication Nos. 20110301073 and 20110145940.

In certain embodiments, the DNA binding domain that binds to a target site a Plasmodium gene comprises a zinc finger protein. Preferably, the zinc finger protein is non-naturally occurring in that it is engineered to bind to a target site of choice. See, for example, See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

An engineered zinc finger binding domain can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, co-owned U.S. Pat. Nos. 6,453,242 and 6,534,261, incorporated by reference herein in their entireties.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237. In addition, enhancement of binding specificity for zinc finger binding domains has been described, for example, in co-owned WO 02/077227.

In addition, as disclosed in these and other references, DNA domains (e.g., multi-fingered zinc finger proteins) may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The zinc finger proteins described herein may include any combination of suitable linkers between the individual zinc fingers of the protein. In addition, enhancement of binding specificity for zinc finger binding domains has been described, for example, in co-owned WO 02/077227.

Selection of target sites; DNA-binding domains and methods for design and construction of fusion proteins (and polynucleotides encoding same) are known to those of skill in the art and described in detail in U.S. Pat. Nos. 6,140,0815; 789,538; 6,453,242; 6,534,261; 5,925,523; 6,007,988; 6,013,453; 6,200,759; WO 95/19431; WO 96/06166; WO 98/53057; WO 98/54311; WO 00/27878; WO 01/60970 WO 01/88197; WO 02/099084; WO 98/53058; WO 98/53059; WO 98/53060; WO 02/016536 and WO 03/016496.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein.

B. Cleavage Domains

Any suitable cleavage domain can be operatively linked to a DNA-binding domain to form a nuclease. For example, ZFP DNA-binding domains have been fused to nuclease domains to create ZFNs—a functional entity that is able to recognize its intended nucleic acid target through its engineered (ZFP) DNA binding domain and cause the DNA to be cut near the ZFP binding site via the nuclease activity. See, e.g., Kim et al. (1996) Proc. Nat'l Acad Sci USA 93(3):1156-1160. More recently, ZFNs have been used for genome modification in a variety of organisms. See, for example, United States Patent Publications 20030232410; 20050208489; 20050026157; 20050064474; 20060188987; 20060063231; and International Publication WO 07/014,275.

As noted above, the cleavage domain may be heterologous to the DNA-binding domain, for example a zinc finger DNA-binding domain and a cleavage domain from a nuclease or a TALEN DNA-binding domain and a cleavage domain, or meganuclease DNA-binding domain and cleavage domain from a different nuclease. Heterologous cleavage domains can be obtained from any endonuclease or exonuclease. Exemplary endonucleases from which a cleavage domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res. 25:3379-3388. Additional enzymes which cleave DNA are known (e.g., S1 Nuclease; mung bean nuclease; pancreatic DNase I; micrococcal nuclease; yeast HO endonuclease; see also Linn et al. (eds.) Nucleases, Cold Spring Harbor Laboratory Press, 1993). One or more of these enzymes (or functional fragments thereof) can be used as a source of cleavage domains and cleavage half-domains.

Similarly, a cleavage half-domain can be derived from any nuclease or portion thereof, as set forth above, that requires dimerization for cleavage activity. In general, two fusion proteins are required for cleavage if the fusion proteins comprise cleavage half-domains. Alternatively, a single protein comprising two cleavage half-domains can be used. The two cleavage half-domains can be derived from the same endonuclease (or functional fragments thereof), or each cleavage half-domain can be derived from a different endonuclease (or functional fragments thereof). In addition, the target sites for the two fusion proteins are preferably disposed, with respect to each other, such that binding of the two fusion proteins to their respective target sites places the cleavage half-domains in a spatial orientation to each other that allows the cleavage half-domains to form a functional cleavage domain, e.g., by dimerizing. Thus, in certain embodiments, the near edges of the target sites are separated by 5-8 nucleotides or by 15-18 nucleotides. However any integral number of nucleotides or nucleotide pairs can intervene between two target sites (e.g., from 2 to 50 nucleotide pairs or more). In general, the site of cleavage lies between the target sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982. Thus, in one embodiment, fusion proteins comprise the cleavage domain (or cleavage half-domain) from at least one Type IIS restriction enzyme and one or more zinc finger binding domains, which may or may not be engineered.

An exemplary Type IIS restriction enzyme, whose cleavage domain is separable from the binding domain, is Fok I. This particular enzyme is active as a dimer. Bitinaite et al. (1998) Proc. Natl. Acad. Sci. USA 95: 10,570-10,575. Accordingly, for the purposes of the present disclosure, the portion of the Fok I enzyme used in the disclosed fusion proteins is considered a cleavage half-domain. Thus, for targeted double-stranded cleavage and/or targeted replacement of cellular sequences using zinc finger-Fok I fusions, two fusion proteins, each comprising a FokI cleavage half-domain, can be used to reconstitute a catalytically active cleavage domain. Alternatively, a single polypeptide molecule containing a DNA binding domain and two Fok I cleavage half-domains can also be used.

A cleavage domain or cleavage half-domain can be any portion of a protein that retains cleavage activity, or that retains the ability to multimerize (e.g., dimerize) to form a functional cleavage domain.

Exemplary Type IIS restriction enzymes are described in International Publication WO 07/014,275, incorporated herein in its entirety. Additional restriction enzymes also contain separable binding and cleavage domains, and these are contemplated by the present disclosure. See, for example, Roberts et al. (2003) Nucleic Acids Res. 31:418-420.

In certain embodiments, the cleavage domain comprises one or more engineered cleavage half-domain (also referred to as dimerization domain mutants) that minimize or prevent homodimerization, as described, for example, in U.S. Patent Publication Nos. 20050064474; 20060188987 and 20080131962, the disclosures of all of which are incorporated by reference in their entireties herein. Amino acid residues at positions 446, 447, 479, 483, 484, 486, 487, 490, 491, 496, 498, 499, 500, 531, 534, 537, and 538 of Fok I are all targets for influencing dimerization of the Fok I cleavage half-domains.

Exemplary engineered cleavage half-domains of Fok I that form obligate heterodimers include a pair in which a first cleavage half-domain includes mutations at amino acid residues at positions 490 and 538 of Fok I and a second cleavage half-domain includes mutations at amino acid residues 486 and 499.

Thus, in one embodiment, a mutation at 490 replaces Glu (E) with Lys (K); the mutation at 538 replaces Iso (I) with Lys (K); the mutation at 486 replaced Gln (Q) with Glu (E); and the mutation at position 499 replaces Iso (I) with Lys (K). Specifically, the engineered cleavage half-domains described herein were prepared by mutating positions 490 (E→K) and 538 (I→K) in one cleavage half-domain to produce an engineered cleavage half-domain designated “E490K:I538K” and by mutating positions 486 (Q→E) and 499 (I→L) in another cleavage half-domain to produce an engineered cleavage half-domain designated “Q486E:I499L”. The engineered cleavage half-domains described herein are obligate heterodimer mutants in which aberrant cleavage is minimized or abolished. See, e.g., U.S. Patent Publication No. 2008/0131962, the disclosure of which is incorporated by reference in its entirety for all purposes.

In certain embodiments, the engineered cleavage half-domain comprises mutations at positions 486, 499 and 496 (numbered relative to wild-type Fold), for instance mutations that replace the wild type. Gln (Q) residue at position 486 with a Glu (E) residue, the wild type Iso (I) residue at position 499 with a Leu (L) residue and the wild-type Asn (N) residue at position 496 with an Asp (D) or Glu (E) residue (also referred to as a “ELD” and “ELE” domains, respectively). In other embodiments, the engineered cleavage half-domain comprises mutations at positions 490, 538 and 537 (numbered relative to wild-type Fold), for instance mutations that replace the wild type Glu (E) residue at position 490 with a Lys (K) residue, the wild type Iso (I) residue at position 538 with a Lys (K) residue, and the wild-type His (H) residue at position 537 with a Lys (K) residue or a Arg (R) residue (also referred to as “KKK” and “KKR” domains, respectively). In other embodiments, the engineered cleavage half-domain comprises mutations at positions 490 and 537 (numbered relative to wild-type Fold), for instance mutations that replace the wild type Glu (E) residue at position 490 with a Lys (K) residue and the wild-type His (H) residue at position 537 with a Lys (K) residue or a Arg (R) residue (also referred to as “KIK” and “KIR” domains, respectively). (See US Publication No. 20110201055).

Engineered cleavage half-domains described herein can be prepared using any suitable method, for example, by site-directed mutagenesis of wild-type cleavage half-domains (Fok I) as described in U.S. Patent Publication Nos. 20050064474; 20080131962 and 20110201055.

Alternatively, nucleases may be assembled in vivo at the nucleic acid target site using so-called “split-enzyme” technology (see, e.g. U.S. Patent Publication No. 20090068164). Components of such split enzymes may be expressed either on separate expression constructs, or can be linked in one open reading frame where the individual components are separated, for example, by a self-cleaving 2A peptide or IRES sequence. Components may be individual zinc finger binding domains or domains of a meganuclease nucleic acid binding domain.

Nucleases can be screened for activity prior to use, for example in a yeast-based chromosomal system as described in WO 2009/042163 and 20090068164. Nuclease expression constructs can be readily designed using methods known in the art. See, e.g., United States Patent Publications 20030232410; 20050208489; 20050026157; 20050064474; 20060188987; 20060063231; and International Publication WO 07/014,275. Expression of the nuclease may be under the control of a constitutive promoter or an inducible promoter, for example the galactokinase promoter which is activated (de-repressed) in the presence of raffinose and/or galactose and repressed in presence of glucose.

### Target Sites

As described in detail above, DNA domains can be engineered to bind to any sequence of choice in a locus, for example a Plasmodium gene. An engineered DNA-binding domain can have a novel binding specificity, compared to a naturally-occurring DNA-binding domain Engineering methods include, but are not limited to, rational design and various types of selection. Rational design includes, for example, using databases comprising triplet (or quadruplet) nucleotide sequences and individual (e.g., zinc finger) amino acid sequences, in which each triplet or quadruplet nucleotide sequence is associated with one or more amino acid sequences of DNA binding domain which bind the particular triplet or quadruplet sequence. See, for example, co-owned U.S. Pat. Nos. 6,453,242 and 6,534,261, incorporated by reference herein in their entireties. Rational design of TAL-effector domains can also be performed. See, e.g., U.S. Patent Publication No. 20110301073.

Exemplary selection methods applicable to DNA-binding domains, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237.

Selection of target sites; nucleases and methods for design and construction of fusion proteins (and polynucleotides encoding same) are known to those of skill in the art and described in detail in U.S. Patent Application Publication Nos. 20050064474 and 20060188987, incorporated by reference in their entireties herein.

In addition, as disclosed in these and other references, DNA-binding domains (e.g., multi-fingered zinc finger proteins) may be linked together using any suitable linker sequences, including for example, linkers of 5 or more amino acids. See, e.g., U.S. Pat. Nos. 6,479,626; 6,903,185; and 7,153,949 for exemplary linker sequences 6 or more amino acids in length. The proteins described herein may include any combination of suitable linkers between the individual DNA-binding domains of the protein. See, also, U.S. Patent Publication No. 20110287512.

### Donors

As noted above, insertion of an exogenous sequence (also called a “donor sequence” or “donor”), for example for correction of a mutant gene or for increased expression of a wild-type gene. It will be readily apparent that the donor sequence is typically not identical to the genomic sequence where it is placed. A donor sequence can contain a non-homologous sequence flanked by two regions of homology to allow for efficient HDR at the location of interest. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest.

The donor polynucleotide can be DNA or RNA, single-stranded or double-stranded and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with an agent such as a liposome or poloxamer.

The donor is generally inserted so that its expression is driven by the endogenous promoter at the integration site, namely the promoter that drives expression of the albumin gene. However, it will be apparent that the donor may comprise a promoter and/or enhancer, for example a constitutive promoter or an inducible or tissue specific promoter.

Furthermore, although not required for expression, exogenous sequences may also be transcriptional or translational regulatory sequences, for example, promoters, enhancers, insulators, internal ribosome entry sites, sequences encoding 2A peptides and/or polyadenylation signals

### Delivery

The nucleases, polynucleotides encoding these nucleases, donor polynucleotides and compositions comprising the proteins and/or polynucleotides described herein may be delivered in vivo or ex vivo by any suitable means.

Methods of delivering nucleases as described herein are described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties.

Nucleases and/or donor constructs as described herein may also be delivered using vectors containing sequences encoding one or more of the zinc finger or TALEN protein(s). Any vector systems may be used including, but not limited to, plasmid vectors. See, also, U.S. Pat. Nos. 6,534,261; 6,607,882; 6,824,978; 6,933,113; 6,979,539; 7,013,219; and 7,163,824, incorporated by reference herein in their entireties. Furthermore, it will be apparent that any of these vectors may comprise one or more of the sequences needed for treatment. Thus, when one or more nucleases and a donor construct are introduced into the cell, the nucleases and/or donor polynucleotide may be carried on the same vector or on different vectors. When multiple vectors are used, each vector may comprise a sequence encoding one or multiple nucleases and/or donor constructs.

Conventional non-viral based gene transfer methods can be used to introduce nucleic acids encoding nucleases and donor constructs in parasitized cells (e.g., Plasmodium-infected mammalian cells) and target tissues. Non-viral vector delivery systems include DNA plasmids, naked nucleic acid, and nucleic acid complexed with a delivery vehicle such as a liposome or poloxamer. For a review of gene therapy procedures, see Anderson, Science 256:808-813 (1992); Nabel & Feigner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Böhm (eds.) (1995); and Yu et al., Gene Therapy 1:13-26 (1994).

Methods of non-viral delivery of nucleic acids include electroporation, lipofection, microinjection, biolistics, virosomes, liposomes, immunoliposomes, polycation or lipid:nucleic acid conjugates, naked DNA, artificial virions, and agent-enhanced uptake of DNA. Also, chemically modified RNAs can be used (See e.g., Komiann et al. (2011) Nature Biotechnology 29(2):154-157).

Additional exemplary nucleic acid delivery systems include those provided by AmaxaBiosystems (Cologne, Germany), Maxcyte, Inc. (Rockville, Md.), BTX Molecular Delivery Systems (Holliston, Mass.) and Copernicus Therapeutics Inc, (see for example US6008336). Lipofection is described in e.g., U.S. Pat. Nos. 5,049,386; 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424, WO 91/16024.

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

Vectors (e.g., retroviruses, adenoviruses, liposomes, etc.) containing nucleases and/or donor constructs can also be administered directly to an organism for transduction of cells in vivo. Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17th ed., 1989).

It will be apparent that the nuclease-encoding sequences and donor constructs can be delivered using the same or different systems. For example, a donor polynucleotide can be carried by a plasmid, while the one or more nucleases can be carried by a AAV vector. Furthermore, the different vectors can be administered by the same or different routes (intramuscular injection, tail vein injection, other intravenous injection, intraperitoneal administration and/or intramuscular injection. The vectors can be delivered simultaneously or in any sequential order.

Formulations for both ex vivo and in vivo administrations include suspensions in liquid or emulsified liquids. The active ingredients often are mixed with excipients that are pharmaceutically acceptable and compatible with the active ingredient. Suitable excipients include, for example, water, saline, dextrose, glycerol, ethanol or the like, and combinations thereof. In addition, the composition may contain minor amounts of auxiliary substances, such as, wetting or emulsifying agents, pH buffering agents, stabilizing agents or other reagents that enhance the effectiveness of the pharmaceutical composition.

The following Examples relate to exemplary embodiments of the present disclosure in which the nuclease comprises a zinc finger nuclease (ZFN). It will be appreciated that this is for purposes of exemplification only and that other nucleases can be used, for instance homing endonucleases (meganucleases) with engineered DNA-binding domains and/or fusions of naturally occurring of engineered homing endonucleases (meganucleases) DNA-binding domains and heterologous cleavage domains or TALENs.

### EXAMPLES

### Example 1

### Design, Construction and General Characterization of Zinc Finger Protein Nucleases (ZFN)

Zinc finger proteins were designed and incorporated into expression vectors for subsequent transfer to P. falciparum expression vectors plasmids essentially as described in Urnov et al. (2005) Nature 435(7042):646-651, Perez et al (2008) Nature Biotechnology 26(7):808-816, and as described in U.S. Pat. No. 6,534,261. Table 1 shows the recognition helices within the DNA binding domain of exemplary ZFPs while Table 2A shows the target sites for these ZFPs, and Table 2B shows the relationship of the two binding sites. Nucleotides in the target site that are contacted by the ZFP recognition helices are indicated in uppercase letters; non-contacted nucleotides indicated in lowercase.

TABLE 1
Plasmodium specific zinc finger nucleases- helix design

| | | Design | | | | | |
|---|---|---|---|---|---|---|---|
| Target | SBS # | F1 | F2 | F3 | F4 | F5 | F6 |
| Pfcrt | 30415 | RQDCLSL | RNDNRKT | TSGSLSR | DRSNLSS | QSSDLSR | NA |
| | | (SEQ ID | (SEQ ID | (SEQ ID | (SEQ ID | (SEQ ID | |
| | | NO: 1) | NO: 2) | NO: 3) | NO: 4) | NO: 5) | |
| Pfcrt | 30413 | QSGNLAR | RQEHRVA | DRSNLSR | DSSARNT | RSDNLSV | TSGSLTR |
| | | (SEQ ID | (SEQ ID | (SEQ ID | (SEQ ID | (SEQ ID | (SEQ ID |
| | | NO: 6) | NO: 7) | NO: 8) | NO: 9) | NO: 10) | NO: 11) |
| Pfcrt | 30414 | RSDNLSV | DRSNLSR | DSSARNT | RSDNLSV | TSGSLTR | NA |
| | | (SEQ ID | (SEQ ID | (SEQ ID | (SEQ ID | (SEQ ID | |
| | | NO: 10) | NO: 8) | NO: 9) | NO: 10) | NO: 11) | |

TABLE 2A
Plasmodium specific ZFNs: Target sites

| | SBS # | SBS | |
|---|---|---|---|
| Target | “abb.” | # | Target site |
| Pfcrt | 30415, “15” | 30415 | tgGCTCACGTTTAGGTGgaggttcttgt |
| | | | (SEQ ID NO: 12) |
| Pfcrt | 30413, “13” | 30413 | ctGTTAAGGTCGACaAGGGAAaaaaaaa |
| | | | (SEQ ID NO: 13) |
| Pfcrt | 30414, “14” | 30414 | ctGTTAAGGTCGACAAGggaaaaaaaaa |
| | | | (SEQ ID NO: 14) |

TABLE 2B
Alignment of binding sites of Plasmodium
specific ZFNs

| | | SEQ |
|---|---|---|
| | | ID |
| Alignment | SBS | NOs |
| TTCCCTtGTCGACCTTAACagatgGCTCACGTTTAGGTG | 30415 | 42 |
| AAGGGAaCAGCTGGAATTGtctacCGAGTGCAAATCCAC | 30413 | 43 |
| CTTGTCGACCTTAACagatgGCTCACGTTTAGGTG | 30415 | 44 |
| GAACAGCTGGAATTGtctacCGAGTGCAAATCCAC | 30414 | 45 |

Note:
binding sites for the ZFNs are underlined.

### Example 2

### ZFN-Mediated Gene Disruption in Plasmodium

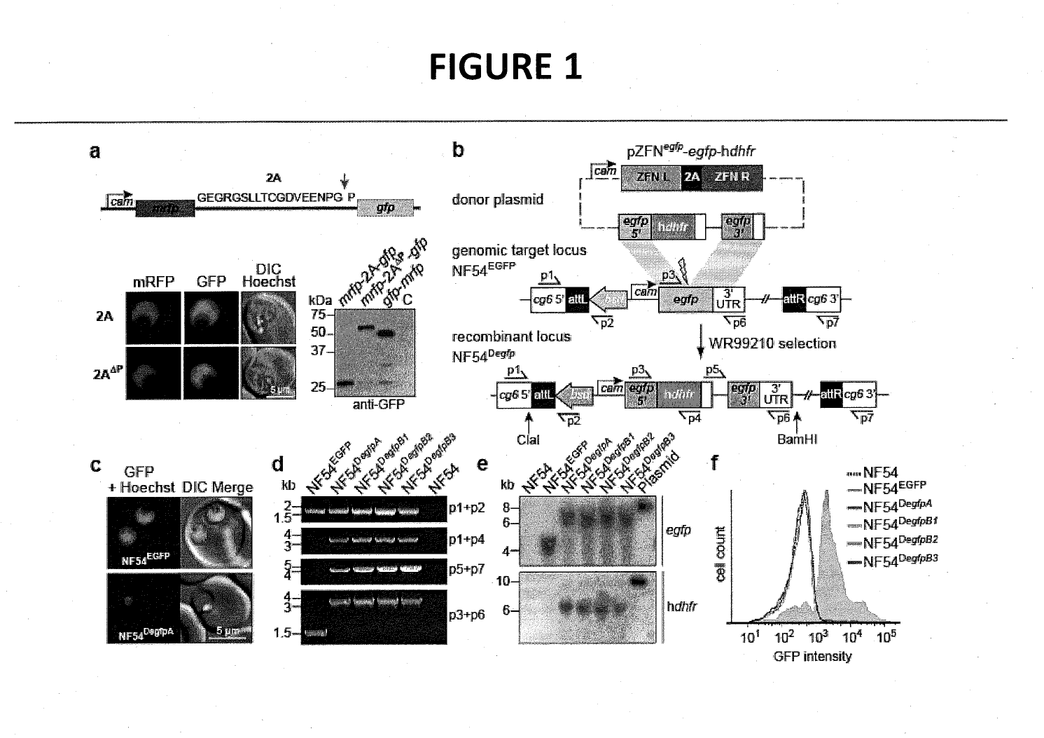

To establish genome editing in P. falciparum, we set out to first establish conditions to optimally express ZFNs; second, determine whether a scorable phenotypic marker could be edited; third, introduce a specific constellation of allelic forms into an endogenous locus relevant to drug resistance. A requirement for directed genome editing is the co-expression within the target cell of two ZFNs that act at the same locus. Due to a dearth of selectable markers for P. falciparum and the instability in Eschericia coli of large plasmids containing AT-rich Plasmodium DNA, we first sought to determine whether a plasmid-encoded ZFN pair could be expressed from a single promoter using the 2A peptide from Thoseaasigna virus (Perez et al, ibid). To assess whether the 2A peptide functions to mediate release of two separate proteins in P. falciparum, we generated transgenic parasites expressing an mRFP-2A-GFP reporter construct driven by the calmodulin (cam) promoter (FIG. 1A). Parasite transfections were performed as described in Fidock and Wellems ((1997) Proc Natl Acad Sci USA 94(20):10931). pZFNegfp-hdhfr was electroporated into NF54eGFP parasites propagated in RPMI-1640 culture medium with 0.5% (w/v) Albumax II (Invitrogen, Carlsbad, Calif.) under 5% O2, 5% CO2, 90% N. Transformed parasites were treated with 2.5 nM WR99210 24 hours (line A), 48 hours (line B1), 96 hours (line B2) and 120 hours (line B3) post transfection to select for parasites with integrated hdhfr. To potentially increase gene disruption efficiency parasite line B was supplemented (1:1) with fresh RBC preloaded with additional plasmid (50 μg) 48 hours post transfection.

Expression of both reporters was detected in parasites by fluorescence microscopy, while immunoblotting for the downstream GFP reporter revealed expression as a 27 kDa monomer. (FIG. 1A). This cotranslational release was crippled by deletion of the P residue at the 2A G-P site, yielding predominantly fused mRFP-2A-GFP product (FIG. 1A).

These findings confirm efficient ribosomal skipping across the 2A site and illustrate its use in P. falciparum to obtain dual protein expression from a single promoter.

To investigate the potential use of ZFN-mediated gene disruption in P. falciparum, we utilized ablation of enhanced green fluorescent protein (eGFP) as an easily quantifiable phenotype. We designed a donor plasmid (termed pZFN^{egfp}-hdhfr) comprising our 2A-linked ZFN expression cassette, as well as two homology regions (denoted egfp 5′ and 3′) that flank the ZFN cut site. Expression of ZFNs in P. falciparum was achieved by cloning egfp ZFNs (Geurts et al, (2009) Science, 325(5939): 433) and pfcrt ZFNs downstream of a calmodulin (cam) promoter and upstream of an hsp86 3′UTR in the pDC2 (Lee et al, (2008) Mol Microbiol 68(6):1535) expression vector. ZFNs linked with the 2A peptide were digested with NheI and XhoI and cloned into the compatible restriction sites AvrII and XhoI in the recipient pDC2 plasmid (Lee, (2008) ibid). To rapidly select for parasites disrupted for the target gene, the egfp 5′ homology region was fused in frame with the human dihydrofolatereductase (hdhfr) selectable marker (Fidock et al (1998) Mol Pharmacol 54:1140), such that resistance to the antifolate drug WR99210 was contingent on integration placing the egfp-hdhfr fusion under the control of the genomic cam promoter (FIG. 1B). Importantly, targeted DHFR ORF addition would also produce a GFP-negative parasite.

To quantify eGFP fluorescence in the parental NF54eGFP and ZFN-modified lines, parasite cultures were analyzed by flow cytometry. Cells were stained for 10 min with 250 nM Syto61 dye (Molecular probes, Invitrogen) in aqueous solution containing 0.2% dextrose and 0.9% sodium chloride. After a single wash 50,000 cells were counted on an Accuri C6 Flow Cytometer. The data was analyzed with FlowJo 7.6.3 gating for nuclear stain Syto61 (FL4) and for green fluorescence (FL1).

We engineered the parasite target strain by integrating the egfp gene into the cg6 locus of a modified NF54 parasite strain (NF54^{attB}) using attB×attP integrase-mediated recombination (Adjalley et al, (2011) Proc Natl Acad Sci USA 108(47) E1214), yielding a uniform population of eGFP-positive parasites (FIGS. 1C and 1D). The resulting parasite line (NF54^{eGFP}) was then transfected with the composite ZFN-donor plasmid (pZFN^{egfp}-hdhfr) and either selected with WR99210 the following day (yielding the parasite line NF54^{eGFP-hDHFR}-A) or supplemented with fresh red blood cells (RBCS) preloaded with additional donor plasmid to potentially increase transfection efficiency (yielding NF54^{eGFP-hDHFR-B1/B2/B3}).

The donor construct containing regions of homology to egfp was generated as follows: oligonucleotides specific to regions adjacent to the predicted ZFN cleavage sites were used to amplify homologous region I (453 bp), denoted egfp 5′ (p3 and p8; Table 3) and homologous region II (795 bp), denoted egfp 3′ (p10 and p11; Table 3). The promoter-less selection cassette hdhfr was amplified with oligonucleotides p9 and p4- and fused in frame to egfp 5′ using overlapping primer (p9 and p8; Table 3 in a splicing by overlap extension PCR reaction.

The fusion construct was cloned in ApaI and SacII restriction sites into pDC2. The second homologous region egfp 3′ was cloned downstream with the restriction sites BstAPI and ZraI. The final plasmid was termed pZFN^{egfp}-hdhfr. P. falciparum trophozoite-infected erythrocytes were harvested and saponin-lysed. Parasite genomic DNA was extracted and purified using DNeasy™ Blood kits (Qiagen). Integration of the hdhfr cassette into the cg6-egfp, locus of NF54^{eGFP} parasites was detected using the primers: i) p1+p2 (specific to cg6 5′UTR and the bsd selectable marker respectively), ii) p1+p4 (specific to cg6 and hdhfr, iii) p5+p7 (specific to the vector backbone and hsp86 3′UTR). iv) p3+p6 (specific to egfp and hsp86 3′UTR). The first primer pair (i) confirms integration of egfp into the cg6 locus for the parental parasite line NF54^{eGFP} as well as for the ZFN transfected parasites NF54^{egfp-hDHFR}-A NF54^{egfp-hDHFR}-B1-3 by amplifying a PCR fragment of 1754 bp. The second primer pair ii) demonstrates disruption of egfp and integration of hdhfr within the cg6 locus upon transfection with pZFNeGFP-hdhfr, amplifying a product of 3883 bp. Reaction iii) yields a product of 4191 by and primer pair iv) produces a 3432 by fragment in transfected parasites and 1478 by in the parental NF54eGFP line. pfcrt gene editing was confirmed by amplifying the genomic locus with p16+p20 located upstream and downstream of the pfcrt donor construct. Sequencing was performed with p12, p13, p17, p18 and p19.

Parasites receiving preloaded RBCs were subjected to drug selection 2-5 days post-transfection. With all four lines, WR99210-resistant parasites were observed on day 15 post-electroporation, and disruption of the egfp gene by integration of hdhfr was confirmed by fluorescence microscopy, PCR and Southern blotting (FIGS. 1C, 1D 1E). Furthermore, flow cytometry revealed a complete loss of eGFP fluorescence in the parasite population, consistent with 100% of the WR99210-resistant parasites carrying the donor-specified ORF at the ZFN target site in the edited genome (FIG. 1D). Flow cytometry revealed the complete loss of fluorescence in all NF54Δegfp lines (FIG. 1F). Three independent transfections with a ZFN-deficient control pegfp-hdhfr plasmid failed to yield parasites after 60 days. Our data illustrate the ability of ZFNs to drive rapid and highly efficient generation of gene knockouts in P. falciparum.

TABLE 3
Oligonucleotides used in study (SEQ ID NOs: 15-41 corresponding to p1 to p27,
respectively)

| | | | Lab |
|---|---|---|---|
| Name | Nucleotide Sequence | Description | Name |
| p1 | GAAAATATTATTACAAAGGGTGAGG | cg6 5′ forward | p1969 |
| p2 | ACGAATTCTTAGCTAATTCGCTTGTAAGA | bsd reverse | p836 |
| p3 | CTGGGCCCATGGTGAGCAAGGGCGAGGAGC | egfp Homologous | p3088 |
| | | region I ApaI | |
| | | forward | |
| p4 | ACCCCGCGGTTAATCATTCTTCTCATATAC | hdhfr-Homologous | p3078 |
| | | region I SacII | |
| | | reverse | |
| p5 | GAGTCGTATTACAATTCACTGG | pDC2 plasmid | p3235 |
| | | backbone Rep20 | |
| p6 | CTTAATCATTTGTATTTGGGAGG | hsp86-3′ reverse | p3202 |
| p7 | CTCTTCTACTCTTTCGAATTC | cg6 reverse | p1970 |
| p8 | CTCCACCGGCGCCAGTAGTAGATCTGGCGGCGGAGAGGG | T2A peptide/mrfp | p3361 |
| | | overlap BglII | |
| | | forward | |
| p9 | CGCGGATCCGCTAGCAGGGCCGGGATTCTCCTCC | T2A peptide NheI- | p3362 |
| | | BamHI reverse | |
| p10 | CGCGGATCCGCTAGCGCCGGGATTCTCCTCC | T2A peptide ΔP21 | p3363 |
| | | NheI-BamHI reverse | |
| p11 | CGCCTAGGATGGCCTCCTCCGAGGACGTCATC | mrfp AvrII forward | p1140 |
| p12 | GGCGCCGGTGGAGTGGC | mrfp reverse | p1141 |
| p13 | TTCCTAGGATGGTGAGCAAGGGCG | egfp AvrII forward | p3006 |
| p14 | CCCTCGAGTTACTTGTACAGCTCGTCC | egfp stop XhI | p3007 |
| | | reverse | |
| p15 | CGAACCAACCATTCCGCTAGCACCGACGTTGTGGCTGTTGTAGTTGTAC | egfp/hdhfr overlap | p3089 |
| | | Homologous Region | |
| | | I reverse | |
| p16 | CAGCCACAACGTCGGTGCTAGCGGAATGGTTGGTTCGCTAAACTGC | hdhfr/egfp overlap | p3090 |
| | | Homologous Region | |
| | | I forward | |
| p17 | CCGCATATGGTGCTATATCATGGCCGACAAGCAGAAG | egfp Homologous | p3091 |
| | | Region II BstAPI | |
| | | forward | |
| p18 | CTGACGTCGAATTTATAAACGTTTGGTTATTAG | hsp86-3′ Homologous | p3080 |
| | | region II ZraI | |
| | | reverse | |
| p19 | GGGCCCCTATAGATTATTTTCATTGTCTTCC | pfcrt 5′UTR | p3128 |
| | | (−150-175 bp) | |
| | | ApaI forward | |
| p20 | CGAGCTCAAGCAGAAGAACATATTAATAGG | pfcrt Exon 3 | p3131 |
| | | SacI reverse | |
| p21 | CGAGATCCATCTATTAGGGTCGAC | pfcrt Intron 1 | p3132 |
| | | ZFN 30413/14/15 | |
| | | mut2 reverse | |
| p22 | CCCTAATAGATGGATCTCGTTTAGG | pfcrt Intron 1 | p3133 |
| | | ZFN 30413/14/15 | |
| | | mut2 forward | |
| p23 | CCAAGTTGTACTGCTTCTAAG | pfcrt 5′UTR | 105′F2 |
| | | (−495-516) | |
| p24 | CCAATAGGTTGATTTATCTATTC | pfcrt Intron 1 | 105′R4 |
| | | reverse | |
| p25 | AGATGGCTCACGTTTAGGTGGAGG | pfcrt Exon 2 | AF12 |
| | | forward | |
| p26 | GTAATGTTTTATATTGGTAGGTGG | pfcrt Exon 2 | 105′R5 |
| | | reverse | |
| p27 | TACAACAATAATAACTGCTCCGAG | pfcrt Exon 4 | AB17B |
| | | reverse | |

To assess potential off-target activity of the ZFNs, we sequenced the genomes of two recombinant lines (NF54^{ΔegfpA} and NF54^{ΔegpB1}) as well as the parent (NF54^{EGFP}). Sequence analysis revealed a depth of coverage of hdhfr (56× and 42× for NF54^{ΔegfpA} and NF54^{ΔegfpB1} respectively) that mirrored the average coverage across the entire genome (54× and 69×), consistent with the presence of a single genomic copy of hdhfr. Furthermore, flanking sequence reads that partially overlapped hdhfr could only be mapped to the egfp-hdhfr locus, consistent with the specific disruption of egfp.

### Example 3

### Gene Replacement in the Absence of a Selectable Phenotype

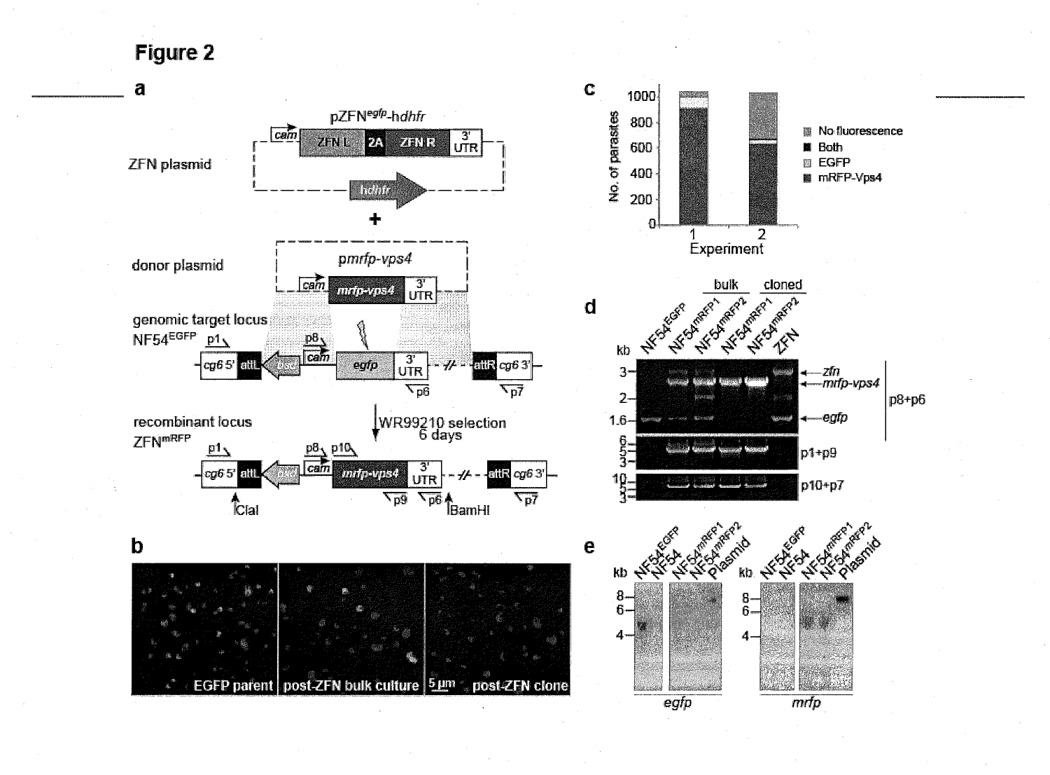

Gene disruption by in-frame integration of a selectable marker is limited to targets that are expressed during the asexual blood stage. We sought to develop a broader strategy for gene manipulation, irrespective of expression pattern during the parasite life cycle and independent of a selection event. We first aimed to replace the egfp reporter with monomeric rfp (mrfp) fused to the cytosolic ATPase pfvps4. This fusion was placed on a donor plasmid (pmrfP-vps4) flanked by egfp untranslated regions (UTRs) and plasmid backbone sequences (3.5 kb and 2.8 kb on the 5′ and 3′ ends respectively). See, FIG. 2A. ZFNs were expressed from a separate plasmid (pZFN^{egfp}-hdhfr) containing the hdhfr selectable marker. The plasmids were co-electroporated, and WR99210 pressure applied for 6 days to transiently enrich for parasites that expressed the ZFNs. Parasite proliferation was detected microscopically 12 days post-electroporation.

Imaging and quantification of parasite fluorescence from the bulk cultures was consistent with a gene replacement efficiency of 88% and 62% in two independent experiments (see, FIGS. 2B and 2C). This level of efficiency was confirmed by analysis of clonal lines, which expressed mRFP and not EGFP in 19/27 (70%) and 21/39 (54%) of cloned parasites from the two experiments. This recombination event involves DNA end resection of greater than 260 by from at least one side of the DSB, leading to invasion of the mrfp flanking sequences common to both the donor plasmid and the chromosomal egfp locus (FIG. 2A). These flanking sequences were shared with the ZFN expression vector, which could compete with the pmrfp-vps4 plasmid as a template for homology-directed repair and could account for the minority of non-fluorescent parasites observed in the bulk cultures (FIG. 2C). PCR and Southern blot analyses confirmed replacement of egfp with the mrfp fusion in the majority of parasites, shown in two representative clones (FIGS. 2D and 2E).

### Example 4

### Allelic Replacement of an Endogenous Parasite Gene

We next sought to utilize ZFNs to engineer a discrete “gene correction” event at an endogenous parasite locus. Unlike conventional allelic replacement strategies for P. falciparum, which typically result in significant modification of the endogenous locus with a selectable marker and other elements of the donor plasmid (van Dijk et al, (2001) Cell 104(1):153), gene correction can deliver as little as a single point mutation to the targeted site from an episomal donor template.

The ability to rapidly generate subtle modifications to the parasite genome has broad utility but is of particular relevance to dissecting drug resistance polymorphisms identified in field and laboratory-based genotyping studies. One of the best-characterized drug resistance determinants in P. falciparum is the chloroquine (CQ) resistance transporter pfcrt, which localizes to the digestive vacuole where hemoglobin degradation and formation of toxic CQ-heme adducts occurs. (Sa et al, (2009) Proc Natl Acad Sci USA 106(45):18883; Fidock et al. (2000) Mol. Cell 6:861-867; Bray et al. (2005) Mol. Microbiol. 56:323-333). Mutant PfCRT mediates resistance to CQ by effluxing drug out of the digestive vacuole. The extensive worldwide use of CQ in malaria treatment has led to the selection of multiple mutations in pfcrt, generating geographically distinct alleles (Summers et al. (2012) Cell Mol. Life Sci. 69:1967-1995). Genetic engineering of isogenic parasites expressing various pfcrt alleles is required to fully analyze their phenotypic impact on drug response, but, to date, this has proven exceptionally time- and labor-intensive. See, Sidhu et al. (2002) Science 298:210-213; Valderramos et al. (2010) PLoS Pathog. 6:e1000887.

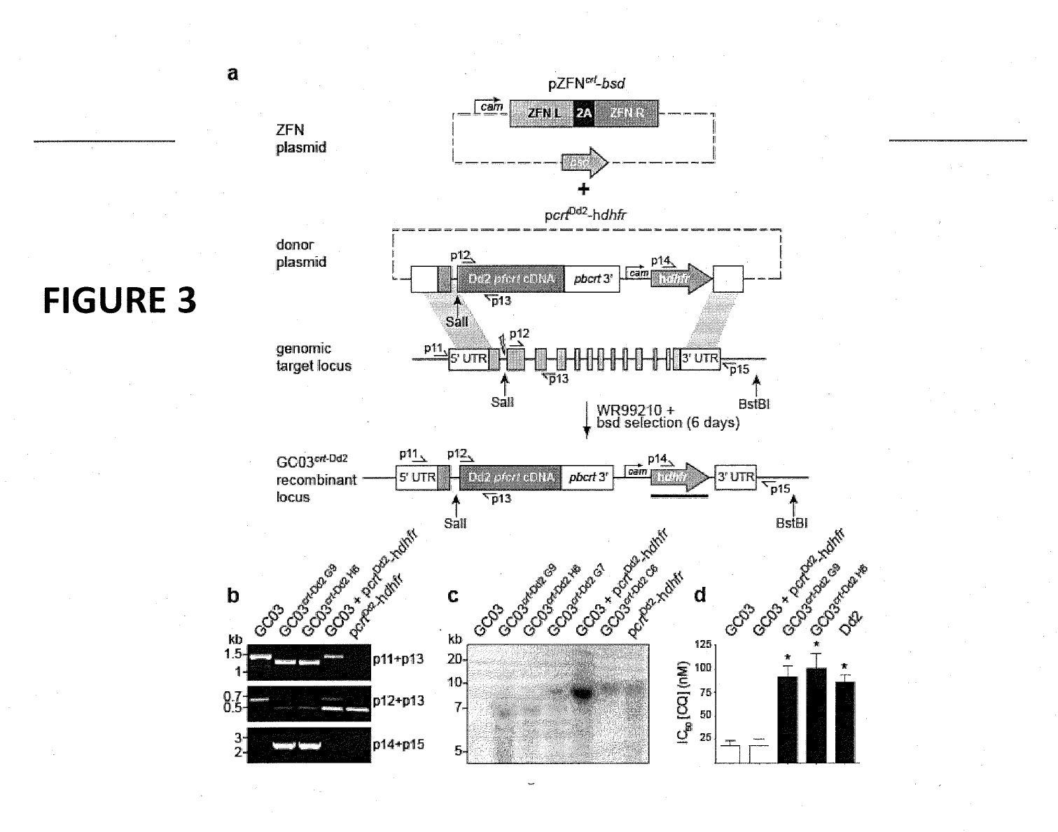

ZFNs were designed as described in Example 1 and tested for activity as described in U.S. Patent Publication 20090111119. The sequences encoding the ZFN pairs shown in Table 1 target the boundary of intron 1 and exon 2, were cloned into a plasmid expressing a blasticidin S-deaminase (bsd) selectable marker, yielding pZFN^{crt}-bsd (FIG. 3A). The pfcrt donor sequence was inserted on a second plasmid (pcrt^{Dd2}-hdhfr), consisting of the pfcrt cDNA from the CQ-resistant (CQR) strain Dd2 and the 3′ UTR from the P. bergheicrt ortholog, followed by a hdhfr expression cassette that served as an independent selectable marker. Upstream and downstream regions of homology, derived from the pfcrt promoter and terminator sequences, flanked these elements to promote ZFN-mediated replacement of the entire 3.1 kb gene with the donor-provided pfcrt 1.2 kb cDNA and the downstream hdhfr selectable marker (FIG. 3A).

We chose to modify the CQ-sensitive (CQS) strains 106/1 and GC03, which harbor distinct alleles and exhibit characteristic drug response phenotypes. Instead of conventional co-transfection, we first electroporated the donor plasmid pcrt^{Dd2}-hdhfr and applied WR99210 to select for episomally transformed parasites (FIG. 3A). These parasites were then electroporated with pZFN^{crt}-bsd, and blasticidin was applied for 6 days to enable transient ZFN expression and consequent homology-directed repair. Prolonged selection for the ZFN plasmid (12 days) caused a delay in parasite re-emergence post-electroporation (data not shown), potentially due to repeated chromosome cleavage. After removal of blasticidin, but not WR99210, parasite proliferation was detected microscopically after 13-16 days.

To quantify the efficiency of pfcrt allelic replacement, clones were generated by limiting dilution and analyzed by PCR. We observed replacement events in 13/82 (15.9%) 106/1 clones and 4/83 (4.8%) GC03 clones (FIG. 3B). Southern blotting of two representative clones (GC03^{crt-Dd2 G9} and GC03^{crt-Dd2 H6}) demonstrated acquisition of the donor-provided CQR pfcrt allele (FIG. 3C). We confirmed the CQ resistance phenotype of these two clones, which both displayed a 4- to 5-fold shift in CQ IC₅₀ values compared to the GC03 parent (FIG. 3D). Notably, in three independent transfections, 106/1 and GC03 parasites that only received the pfcrt donor plasmid but not the ZFN plasmid failed to yield allelic replacement parasites after more than 6 months.

### Example 5

### Site-Specific Editing of a Parasite Drug-Resistance Locus

We next assessed whether our engineered pfcrt-targeted ZFNs could drive a subtle gene-editing event that delivers a single point mutation to the targeted site from an episomal donor template. In contrast, conventional allelic exchange strategies for P. falciparum typically result in significant modification of the endogenous locus by crossover-mediated incorporation of the entire plasmid (often as a concatamer), including a selectable marker and other sequence elements.

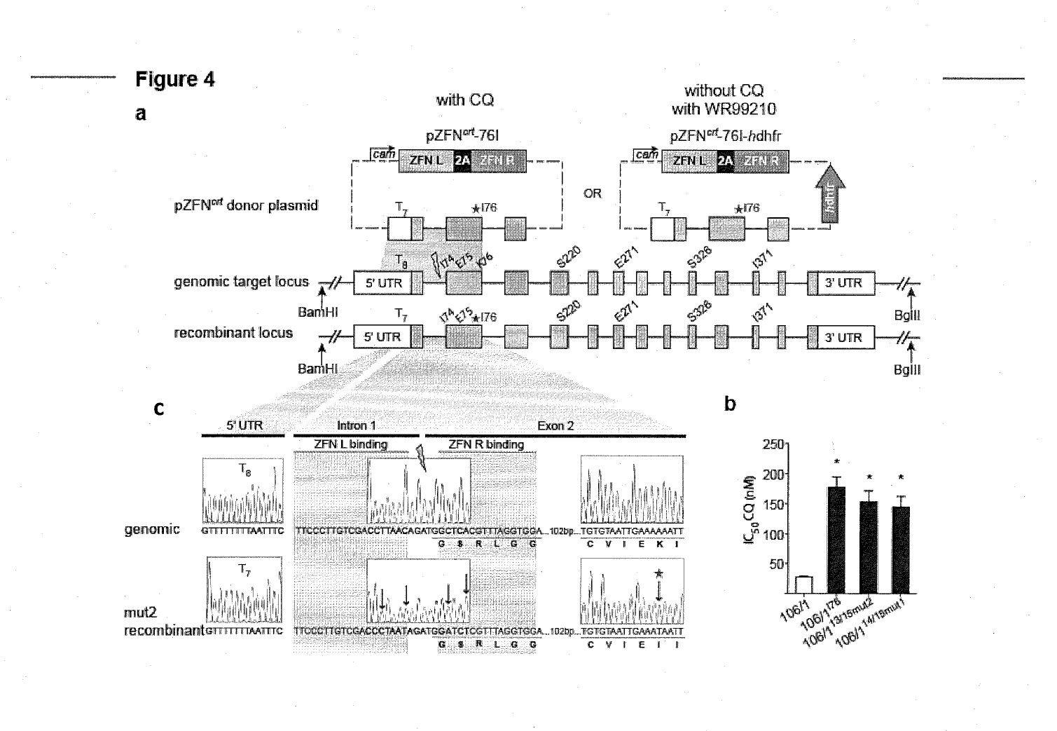

To achieve gene editing in P. falciparum, we exploited the CQ resistance-conferring properties of mutant pfcrt. PfCRT mediates resistance by effluxing CQ from the digestive vacuole, dependent on mutation of residue K76 to T (in the case of field isolates) or I (observed in CQ-pressured 106/1 parasites, see, e.g., Fidock et al. (2000) ibid, Cooper et al. (2003) ibid, Martin et al. (2009) Science 325:1680-1682). pfcrt alleles from CQR parasite strains also possess at least 3 additional, potentially compensatory mutations (Elliot et al. (1998) Mol. Cell. Biol. 57:93-101). As described in Example 4, the CQ-sensitive (CQS) Sudanese isolate 106/1 was used, as its pfcrt allele encodes six out of seven CQR mutations observed in Asian and African strains while retaining the CQS K76 codon (FIG. 4A). All donor sequences provided for the ZFN induced DSB repair were placed on the same plasmid as the ZFN expression cassette. Based on prior selection studies (Cooper et al; (2002) Mol. Pharmacol 61(1):35, Fidock, (2000) ibid), editing of the K76 codon to I in this isolate was predicted to establish a CQ resistance phenotype.

A pfcrt 1 kb donor sequence harboring the K76I mutation and spanning this targeted codon was inserted into the ZFN expression plasmid (FIG. 4A). We tested two versions of the donor sequence: one with an intact ZFN binding site (“mut1”), and another with four silent mutations (“mut2”). The latter was designed to prevent ZFN binding and cleavage of a successfully modified chromosomal target, thereby potentially enhancing editing efficiency. The donor construct used for gene editing of pfcrt was generated as follows: a PCR fragment encompassing 400 by upstream and 600 by downstream of the predicted ZFN target site at the intron 1-exon 2 boundary was amplified from gDNA isolated from 106/176I (Fidock, (2000) ibid, Cooper, (2002) ibid) using oligonucleotides p12 and p13. 106/176I was derived by drug selection from 106/1 and contains all seven CQ resistance mutations. The hdhfr selection cassette of pDC2 was excised with Apal and Sad and replaced by the pfcrt donor fragment (termed ‘mut1’). A second donor template was generated which contained four silent mutations at the predicted ZFN binding site to prevent repeated cleavage. These SNPs were introduced via splicing by overlap extension PCR using primer p12+p14 and p13+p15 in the first reaction and p12+p 13 in the nested PCR reaction (Table 3). The resulting fragment was termed ‘mut2’ and cloned as the ‘mut1’ donor above. Both ZFN pairs (13/15 and 14/15) were expressed from a plasmid containing either the “mut-1” or “mut-2” donor. Accordingly plasmids were termed pZFNpfcrt13/15-mut1, pZFNpfcrt14/15-mut1, pZFNpfcrt13/15-mut2 and pZFNpfcrt14/15-mut2. pZFN^{pfct} with either the mut1 or mut2 donor were electroporated into the CQS strain 106/1 that contains six out of seven CQ-resistant mutations.

Transfected 106/1 parasites were pressured the following day with 33 nM CQ, a concentration sufficient to kill the CQS parent line but significantly below the IC₅₀ values of at least 80-100 nM that typify in vitro CQ resistance. Microscopic assessment of blood smears revealed parasite proliferation under CQ pressure 16 to 33 days post-electroporation (Table 4). In contrast, similar CQ exposure of six independent non-transfected 106/1 cultures, beginning with parasite numbers equivalent to those used for ZFN-mediated gene editing), yielded no parasites after 90 days.

To confirm acquisition of the K76I mutation, we PCR amplified the pfcrt locus using primers external to the donor template and subcloned these products for sequence analysis. In five independent parasite transfections, we observed 100% K76I conversion rates (FIG. 4; Table 4).

TABLE 4
ZFN-mediated gene editing of pfcrt either with or without selection

| | | | | | Successful editing event | | |
|---|---|---|---|---|---|---|---|
| | | | | | Binding | | |
| | | | | Sequences | site | | T |
| | Strain | ZFN pair | Donor | analyzed | mutations | K76I | (deletion) |
| CQ | 106/1 | 13/15 | pcrt-76I-mut1 | 29 pGEM-T | N/A | 29/29 | 0/29 |
| | 106/1 | 13/15 | pcrt-76I-mut2 | 25 pGEM-T | 25/25 | 25/25 | 9/25 |
| | 106/1 | 14/15 | pcrt-76I-mut1 | 38 pGEM-T | N/A | 38/38 | 38/38 |
| | 106/1 | 14/15 | pcrt-76I-mut1 | 28 pGEM-T | N/A | 28/28 | 28/28 |
| | 106/1 | 14/15 | pcrt-76I-mut2 | 31 pGEM-T | 31/31 | 31/31 | 6/31 |
| Targeting efficiency with CQ selection: | | | | | 100% | 100% | 51% |
| no CQ | Dd2 | 13/15 | pcrt-76I-mut2 | 36 parasite | 4/36 | 2/36 | 4/4* |
| | | | | clones | | | |
| | Dd2 | 14/15 | pcrt-76I-mut2 | 40 parasite | 10/40 | 10/40 | 10/10* |
| | | | | clones | | | |

| | | | |
|---|---|---|---|
| Targeting efficiency without CQ selection: | 18.4% | 15.8% | (18.4%) |
| Distance from ZFN cut site: | 3-6 bp | 140 bp | 296 bp |

No alternate mutations were detected at the K76 codon, in particular the K76T mutation commonly found in the vast majority of CQR parasites. Editing of the K76 codon occurred efficiently using either ZFN pair (13/15 or 14/15), and regardless of whether the ZFN binding site was mutated in the donor construct (“mut1” or “mut2”; Table 4). Notably, the additional 4 silent mutations in the “mut2” template were also incorporated at the pfcrt locus, in agreement with the notion that gene correction proceeds via SDSA. In addition, both the “mut1” and “mut2” donor templates carried a small indel (the deletion of a single bp, i.e., a string of seven Ts (T₇), compared to T₈ in the endogenous locus) in the 5′ untranslated region of pfcrt, located ˜300 by upstream of the ZFN cut site. This deletion, located ˜300 by upstream of the ZFN cut site, was transferred into the edited gene sequence with a mean efficiency of 51% (Table 4). By comparison, mutations located an equivalent distance from the ZFN cleavage site have been captured with considerably lower frequency in mammalian cells (e.g. 5% in mouse embryonic stems cells). Importantly, the T₇ deletion was captured despite its presence on the side opposite the DSB relative to the selected K76I mutation. Incorporation into the chromosomal target of all mutations on the donor plasmid could be explained by gene editing proceeding via synthesis-dependent strand annealing or other non-crossover events (FIG. 4).

We also confirmed the CQ resistance phenotype of two gene-edited lines, 106/1^{13/15mut2} and 106/1^{14/15mut1} (FIG. 4A). Briefly, in vitro IC₅₀ values were determined by incubating the CQ resistant parasites 106/1^{76I}, 106/1^{14/15-mut1} and 106/1^{13/15-mut2} for 72 h across a range of concentrations of CQ diphosphate (2000 nM-3.9 nM) and the parental CQS parasite 106/1 to 10 concentrations covering a range of 200 nM-2.5 nM. Parasitemia was determined by flow cytometry after a 72 h incubation with drug. Parasites were stained with 1.6 μM Mito Tracker® (Molecular probes, Invitrogen) and 2×Sybr® Green (Molecular probes, Invitrogen) in 1×PBS supplemented with 5% FBS as described. In vitro IC₅₀ values were calculated by non-linear regression analysis and Mann-Whitney U tests were employed for statistical analysis.

Both lines displayed a 5-6 fold shift in CQ IC₅₀ values relative to the unmodified 106/1 parent line. This shift in drug response was comparable to a CQ resistant line of 106/1 (106/1^{76I}) bearing the equivalent K76I mutation that was previously derived by drug selection (Fidock, 2000 ibid; Cooper, 2002 ibid).

Whole-genome sequencing revealed no detectable off-target activity of the pfcrt-targeting ZFN pairs in two representative recombinant lines (106/1^{13/15mut1} and 106/1^{14/15mut1}). Illumina next-generation sequencing yielded a 15× coverage for >97% of all three genomes. We found no evidence of any rearrangement of the pfcrt locus in these edited lines, and confirmed 100% incorporation of the K76I mutation.

To demonstrate the applicability of ZFNs to generate SNPs that may not confer a selectable phenotype, we repeated the ZFN-mediated pfcrt K76I editing event described above without applying CQ pressure. To select for transfected parasites and ensure ZFN expression, we added the hdhfr selection cassette to the mut2 version of the pZFN^{crt}-76I plasmid (yielding pZFN^{crt}-76I-hdhfr) (FIG. 4A). Transfected Dd2 parasites were selected with WR99210 for 6 days, and parasite proliferation was observed 11 days after removal of drug. From two independent experiments, we generated a total of 76 clones and used these to PCR-amplify the pfcrt genomic locus.

This analysis identified the ZFN binding site mutations in 18.4% and the K76I mutation in 15.8% of clones. The upstream T₇ deletion was also found in all edited clones. These data suggest that non-selected gene editing events can be generated with sufficient efficiency to readily permit the isolation of modified parasite clones by limiting dilution, thus expanding the range of potential targets beyond those related to drug resistance.

Thus ZFN-induced gene editing of an endogenous parasite gene can rapidly generate a panel of lines to assess the impact of precise, user-defined genotypic changes on parasite phenotype.

All patents, patent applications and publications mentioned herein are hereby incorporated by reference in their entirety.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

What is claimed is:

1. A zinc finger DNA-binding domain comprising five or six zinc finger recognition regions designated and ordered F1 to F5 or F1 to F6 as shown in a single row of Table 1, wherein the zinc finger DNA-binding domain binds to a target site in an endogenous Plasmodium gene.

2. A fusion protein comprising the zinc finger DNA-binding domain of claim 1 and a cleavage domain or cleavage half-domain.

3. A polynucleotide comprising a sequence encoding a polypeptide according to claim 1.

4. A gene delivery vector comprising a polynucleotide according to claim 3.

5. An isolated cell comprising a polypeptide according to claim 1.

6. A method of inactivating one or more Plasmodium genes in a Plasmodium spp., the method comprising:
cleaving the one or more Plasmodium genes using one or more fusion proteins according to claim 2 in the presence of an exogenous donor sequence such that the exogenous donor sequence is integrated via homology-directed repair into the one or more cleaved Plasmodium genes, wherein integration of the exogenous donor inactivates the one or more Plasmodium genes.

7. The method of claim 6, wherein the exogenous donor sequence inactivates the one or more Plasmodium genes by creating an insertion or deletion in the one or more Plasmodium genes.

8. A method of inhibiting Plasmodium spp. invasion of or replication within a cell, the method comprising:
inactivating one or more Plasmodium genes in a Plasmodium spp. according to the method of claim 6, thereby inhibiting Plasmodium spp. invasion of or replication within a cell.

9. The method of claim 8, wherein the cell is a blood cell or a liver cell.

10. A method for generating an immune response against a Plasmodium spp. in a subject, the method comprising:
inactivating one or more Plasmodium genes in a Plasmodium spp. according to the method of claim 6; and
administering the Plasmodium spp. to the subject.

11. The method of claim 10, wherein the immune response treats or prevents malarial infection in the subject.

12. A Plasmodium spp. in which one or more endogenous genes are inactivated according to the method of claim 6.


SEQUENCE LISTING (55 sequences)

SEQ ID NO: 1 - protein, 7 aa, Artificial Sequence
  Description of Artificial Sequence Synthetic peptide
RQDCLSL

SEQ ID NO: 2 - protein, 7 aa, Artificial Sequence
  Description of Artificial Sequence Synthetic peptide
RNDNRKT

SEQ ID NO: 3 - protein, 7 aa, Artificial Sequence
  Description of Artificial Sequence Synthetic peptide
TSGSLSR

SEQ ID NO: 4 - protein, 7 aa, Artificial Sequence
  Description of Artificial Sequence Synthetic peptide
DRSNLSS

SEQ ID NO: 5 - protein, 7 aa, Artificial Sequence
  Description of Artificial Sequence Synthetic peptide
QSSDLSR

SEQ ID NO: 6 - protein, 7 aa, Artificial Sequence
  Description of Artificial Sequence Synthetic peptide
QSGNLAR

SEQ ID NO: 7 - protein, 7 aa, Artificial Sequence
  Description of Artificial Sequence Synthetic peptide
RQEHRVA

SEQ ID NO: 8 - protein, 7 aa, Artificial Sequence
  Description of Artificial Sequence Synthetic peptide
DRSNLSR

SEQ ID NO: 9 - protein, 7 aa, Artificial Sequence
  Description of Artificial Sequence Synthetic peptide
DSSARNT

SEQ ID NO: 10 - protein, 7 aa, Artificial Sequence
  Description of Artificial Sequence Synthetic peptide
RSDNLSV

SEQ ID NO: 11 - protein, 7 aa, Artificial Sequence
  Description of Artificial Sequence Synthetic peptide
TSGSLTR

SEQ ID NO: 12 - DNA, 28 bp, Plasmodium sp.
tggctcacgtttaggtggaggttcttgt

SEQ ID NO: 13 - DNA, 28 bp, Plasmodium sp.
ctgttaaggtcgacaagggaaaaaaaaa

SEQ ID NO: 14 - DNA, 28 bp, Plasmodium sp.
ctgttaaggtcgacaagggaaaaaaaaa

SEQ ID NO: 15 - DNA, 25 bp, Artificial Sequence
  Description of Artificial Sequence Synthetic oligonucleotide
gaaaatattattacaaagggtgagg

SEQ ID NO: 16 - DNA, 29 bp, Artificial Sequence
  Description of Artificial Sequence Synthetic oligonucleotide
acgaattcttagctaattcgcttgtaaga

SEQ ID NO: 17 - DNA, 30 bp, Artificial Sequence
  Description of Artificial Sequence Synthetic oligonucleotide
ctgggcccatggtgagcaagggcgaggagc

SEQ ID NO: 18 - DNA, 30 bp, Artificial Sequence
  Description of Artificial Sequence Synthetic oligonucleotide
accccgcggttaatcattcttctcatatac

SEQ ID NO: 19 - DNA, 22 bp, Artificial Sequence
  Description of Artificial Sequence Synthetic oligonucleotide
gagtcgtattacaattcactgg

SEQ ID NO: 20 - DNA, 23 bp, Artificial Sequence
  Description of Artificial Sequence Synthetic oligonucleotide
cttaatcatttgtatttgggagg

SEQ ID NO: 21 - DNA, 21 bp, Artificial Sequence
  Description of Artificial Sequence Synthetic oligonucleotide
ctcttctactctttcgaattc

SEQ ID NO: 22 - DNA, 39 bp, Artificial Sequence
  Description of Artificial Sequence Synthetic oligonucleotide
ctccaccggcgccagtagtagatctggcggcggagaggg

SEQ ID NO: 23 - DNA, 34 bp, Artificial Sequence
  Description of Artificial Sequence Synthetic oligonucleotide
cgcggatccgctagcagggccgggattctcctcc

SEQ ID NO: 24 - DNA, 31 bp, Artificial Sequence
  Description of Artificial Sequence Synthetic oligonucleotide
cgcggatccgctagcgccgggattctcctcc

SEQ ID NO: 25 - DNA, 32 bp, Artificial Sequence
  Description of Artificial Sequence Synthetic oligonucleotide
cgcctaggatggcctcctccgaggacgtcatc

SEQ ID NO: 26 - DNA, 17 bp, Artificial Sequence
  Description of Artificial Sequence Synthetic oligonucleotide
ggcgccggtggagtggc

SEQ ID NO: 27 - DNA, 24 bp, Artificial Sequence
  Description of Artificial Sequence Synthetic oligonucleotide
ttcctaggatggtgagcaagggcg

SEQ ID NO: 28 - DNA, 27 bp, Artificial Sequence
  Description of Artificial Sequence Synthetic oligonucleotide
ccctcgagttacttgtacagctcgtcc

SEQ ID NO: 29 - DNA, 49 bp, Artificial Sequence
  Description of Artificial Sequence Synthetic oligonucleotide
cgaaccaaccattccgctagcaccgacgttgtggctgttgtagttgtac

SEQ ID NO: 30 - DNA, 46 bp, Artificial Sequence
  Description of Artificial Sequence Synthetic oligonucleotide
cagccacaacgtcggtgctagcggaatggttggttcgctaaactgc

SEQ ID NO: 31 - DNA, 37 bp, Artificial Sequence
  Description of Artificial Sequence Synthetic oligonucleotide
ccgcatatggtgctatatcatggccgacaagcagaag

SEQ ID NO: 32 - DNA, 33 bp, Artificial Sequence
  Description of Artificial Sequence Synthetic oligonucleotide
ctgacgtcgaatttataaacgtttggttattag

SEQ ID NO: 33 - DNA, 31 bp, Artificial Sequence
  Description of Artificial Sequence Synthetic oligonucleotide
gggcccctatagattattttcattgtcttcc

SEQ ID NO: 34 - DNA, 30 bp, Artificial Sequence
  Description of Artificial Sequence Synthetic oligonucleotide
cgagctcaagcagaagaacatattaatagg

SEQ ID NO: 35 - DNA, 24 bp, Artificial Sequence
  Description of Artificial Sequence Synthetic oligonucleotide
cgagatccatctattagggtcgac

SEQ ID NO: 36 - DNA, 25 bp, Artificial Sequence
  Description of Artificial Sequence Synthetic oligonucleotide
ccctaatagatggatctcgtttagg

SEQ ID NO: 37 - DNA, 21 bp, Artificial Sequence
  Description of Artificial Sequence Synthetic oligonucleotide
ccaagttgtactgcttctaag

SEQ ID NO: 38 - DNA, 23 bp, Artificial Sequence
  Description of Artificial Sequence Synthetic oligonucleotide
ccaataggttgatttatctattc

SEQ ID NO: 39 - DNA, 24 bp, Artificial Sequence
  Description of Artificial Sequence Synthetic oligonucleotide
agatggctcacgtttaggtggagg

SEQ ID NO: 40 - DNA, 24 bp, Artificial Sequence
  Description of Artificial Sequence Synthetic oligonucleotide
gtaatgttttatattggtaggtgg

SEQ ID NO: 41 - DNA, 24 bp, Artificial Sequence
  Description of Artificial Sequence Synthetic oligonucleotide
tacaacaataataactgctccgag

SEQ ID NO: 42 - DNA, 39 bp, Plasmodium sp.
ttcccttgtcgaccttaacagatggctcacgtttaggtg

SEQ ID NO: 43 - DNA, 39 bp, Plasmodium sp.
cacctaaacgtgagccatctgttaaggtcgacaagggaa

SEQ ID NO: 44 - DNA, 35 bp, Plasmodium sp.
cttgtcgaccttaacagatggctcacgtttaggtg

SEQ ID NO: 45 - DNA, 35 bp, Plasmodium sp.
cacctaaacgtgagccatctgttaaggtcgacaag

SEQ ID NO: 46 - protein, 9 aa, Unknown
  Description of Unknown “LAGLIDADG” family peptide motif sequence
LAGLIDADG

SEQ ID NO: 47 - protein, 19 aa, Plasmodium falciparum
GEGRGSLLTCGDVEENPGP

SEQ ID NO: 48 - DNA, 56 bp, Plasmodium falciparum
  CDS (39)..(56)
gttttttttaatttcttcccttgtcgaccttaacagatggctcacgtttaggtgga

SEQ ID NO: 49 - protein, 6 aa, Plasmodium falciparum
GSRLGG

SEQ ID NO: 50 - DNA, 18 bp, Plasmodium falciparum
  CDS (1)..(18)
tgtgtaattgaaaaaatt

SEQ ID NO: 51 - protein, 6 aa, Plasmodium falciparum
CVIEKI

SEQ ID NO: 52 - DNA, 55 bp, Artificial Sequence
  Description of Artificial Sequence Synthetic oligonucleotide
gtttttttaatttcttcccttgtcgaccctaatagatggatctcgtttaggtgga

SEQ ID NO: 53 - protein, 6 aa, Artificial Sequence
  Description of Artificial Sequence Synthetic peptide
GSRLGG

SEQ ID NO: 54 - DNA, 18 bp, Artificial Sequence
  Description of Artificial Sequence Synthetic oligonucleotide
tgtgtaattgaaataatt

SEQ ID NO: 55 - protein, 6 aa, Artificial Sequence
  Description of Artificial Sequence Synthetic peptide
CVIEII